(19) United States
(12) Patent Application Publication
(10) Pub. No.: US 2026/0253749 A1
(43) Pub. Date: Aug. 27, 2026

(54) SYSTEM

(71) Applicant: SoftBank Group Corp., Tokyo (JP)

(72) Inventor: Eiji IKEDA, Tokyo (JP)

(73) Assignee: SoftBank Group Corp., Tokyo (JP)

(21) Appl. No.: 19/537,596

(22) Filed: Feb. 12, 2026

(30) Foreign Application Priority Data
Feb. 21, 2025 (JP) 2025-026995

(51) Int. Cl. G16H 80/00; A61B 5/00; A61B 5/16; G10L 15/16; G10L 15/18; G10L 15/183; G10L 25/63; G10L 25/66

(52) U.S. Cl. CPC G16H 80/00; A61B 5/0022; A61B 5/165; A61B 5/4803; G10L 15/16; G10L 25/63; G10L 25/66; G10L 15/1815; G10L 15/183

(57) ABSTRACT

The system according to the embodiment comprises an operation support unit, a conversation support unit, a health monitoring unit, an abnormality detection unit, and a cooperation unit. The operation support unit provides operation support. The conversation support unit provides conversation support based on an operation supported by the operation support unit. The health monitoring unit monitors a health condition based on a conversation supported by the conversation support unit. The abnormality detection unit detects an abnormality based on the health condition monitored by the health monitoring unit. The cooperation unit cooperates with a care taxi or medical system based on the abnormality detected by the abnormality detection unit.

### CROSS-REFERENCE TO RELATED APPLICATION

The present application claims priority to and incorporates by reference the entire contents of Japanese Patent Application No. 2025-026995 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, it has been difficult to efficiently provide life support and health management for elderly people, and there is room for improvement.

### SUMMARY OF THE INVENTION

The system according to the embodiment comprises an operation support unit, a conversation support unit, a health monitoring unit, an abnormality detection unit, and a cooperation unit. The operation support unit provides operation support. The conversation support unit provides conversation support based on an operation supported by the operation support unit. The health monitoring unit monitors a health condition based on a conversation supported by the conversation support unit. The abnormality detection unit detects an abnormality based on the health condition monitored by the health monitoring unit. The cooperation unit cooperates with a care taxi or medical system based on the abnormality detected by the abnormality detection unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

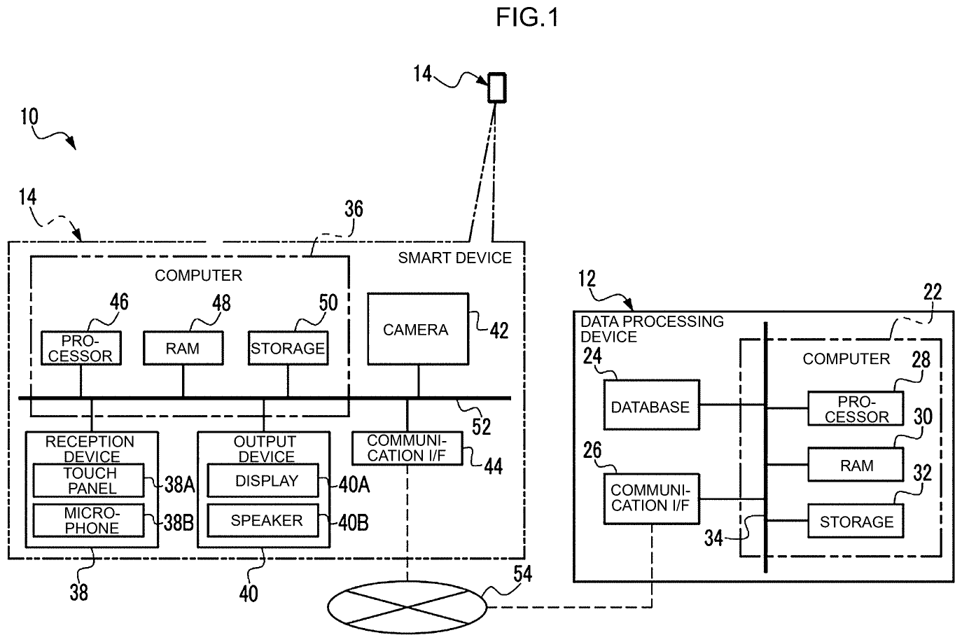

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

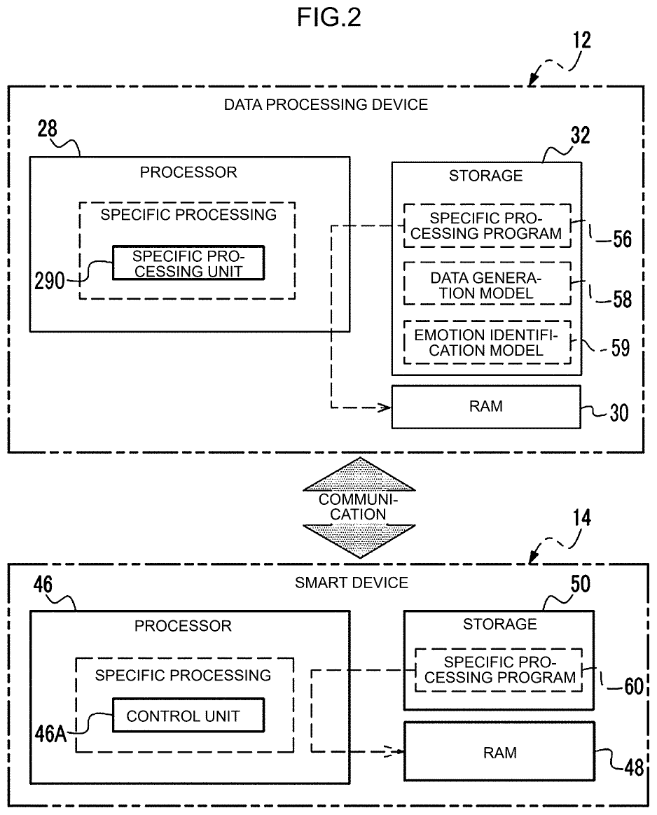

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The care support system according to the embodiment of the present invention is a system that utilizes generative AI to support the daily life and safety of elderly persons and assist in health management. This care support system secures the daily life and safety of elderly persons and supports health management by using robot companions and monitoring systems. For example, the robot companion supports the daily life of elderly persons, such as assisting with meal preparation, providing medication reminders, and engaging in daily conversation. Next, the monitoring system constantly monitors the health condition of elderly persons and issues alerts when abnormalities are detected. For example, if an abnormality in heart rate or blood pressure is detected, emergency contact is made with family members or medical institutions. Furthermore, by cooperating with care taxis and medical systems and using voice guidance, the system predicts signs of serious illness from the condition of the elderly person and performs necessary treatment and emergency contact with family members. As a result, the quality of life of elderly persons can be improved, and a safe and healthy life can be supported. For example, the robot companion supports the daily life of elderly persons, such as assisting with meal preparation and providing medication reminders. Additionally, as a conversation partner, the robot companion can alleviate the sense of loneliness of elderly persons. Furthermore, the robot companion supports the movements of elderly persons and can reduce the risk of falls, such as by providing walking assistance and standing-up support. Next, the monitoring system constantly monitors the health condition of elderly persons, such as monitoring vital signs (heart rate, blood pressure, body temperature) in real time and issuing alerts when abnormalities are detected. This enables constant understanding of the health condition of elderly persons and early detection of abnormalities. The monitoring system also learns behavioral patterns of elderly persons and issues alerts when abnormal behavior is detected, such as not waking up at the usual time or remaining inactive for a long period. Furthermore, by cooperating with care taxis and medical systems and using voice guidance, the system predicts signs of serious illness from the condition of the elderly person. For example, the voice guidance can analyze breathing sounds and coughing sounds of elderly persons to detect signs of pneumonia, and can also analyze speech content to detect signs of dementia, enabling early and appropriate treatment. In this way, the care support system utilizing generative AI can support the daily life and safety of elderly persons and assist in health management, thereby improving their quality of life and supporting a safe and healthy lifestyle. Specifically, this care support system achieves advanced information processing beyond conventional simple automation by linking multiple AI modules. The system comprises multiple hardware and software modules, such as a robot companion unit, monitoring unit, voice analysis unit, abnormality detection unit, and cooperation unit. The robot companion unit is equipped with a deep learning-based natural language processing model (e.g., transformer-based large language model), receives voice input (e.g., 16 kHz sampled 1D waveform data, 10 seconds in length, content such as “Good morning” or “I'm not feeling well today”), and after converting to text, generates conversation or reminders. The output consists of text data for speech synthesis and behavioral instruction commands (e.g., “prompt meal preparation,” “issue medication reminder”). The monitoring unit receives vital signs obtained from wearable sensors (e.g., heart rate 60-120 bpm, blood pressure 100-180 mmHg, body temperature 35-39° C. as time-series array data, updated every minute) as input and notifies the abnormality detection unit of threshold exceedances or pattern abnormalities (e.g., heart rate exceeding 120 bpm, body temperature rising to 39° C.). The abnormality detection unit uses a supervised learning abnormality detection model (e.g., LSTM-based time-series abnormality detection network) to analyze input vital signs and behavioral patterns (e.g., delayed wake-up time, decreased activity) as multidimensional vectors and outputs abnormality scores (e.g., 0.85, exceeding the abnormality threshold of 0.7) and abnormality labels (e.g., “high risk,” “caution required”). The voice analysis unit inputs spectrogram-transformed breathing and coughing sounds (e.g., 1-second mel spectrogram, 128 dimensions×100 frames) into a CNN-based acoustic abnormality detection model and outputs abnormality probabilities (e.g., 0.92 abnormal, 0.08 normal) and abnormality types (e.g., “wheezing,” “wet cough”). These outputs are sent by the cooperation unit to care taxi dispatch APIs and medical institution notification APIs, and push notifications are sent to family members or medical professionals as needed. Furthermore, the system accumulates behavioral history and health data of elderly persons in a time-series database, enabling early detection of abnormalities and preventive intervention by comparing with past data and analyzing trends. For AI model training, transfer learning and data augmentation (e.g., noise addition, voice pitch conversion) are used to achieve high-accuracy inference even with small amounts of data. Unlike conventional human observation or rule-based processing, this system performs pattern extraction in high-dimensional feature space and integrated analysis of multiple sensor information, greatly improving accuracy, speed, and reproducibility. Technical effects include earlier abnormality detection, reduced false alarm rates, reduced burden on care sites, and realization of remote health management. Application fields include home care, care facilities, medical institutions, remote monitoring services, and rehabilitation support.

The care support system according to the embodiment comprises an operation support unit, a conversation support unit, a health monitoring unit, an abnormality detection unit, and a cooperation unit. The operation support unit supports the movements of elderly persons. For example, the operation support unit provides walking assistance, such as supporting the walking of elderly persons using a walker. The operation support unit also provides standing-up support, such as supporting the standing-up of elderly persons using a standing-up assistance device. Furthermore, the operation support unit may use robots to support the movements of elderly persons. For example, the operation support unit supports the movements of elderly persons using a robot. The conversation support unit provides conversation support based on the movements supported by the operation support unit. For example, the conversation support unit alleviates loneliness by engaging in conversation with elderly persons while they are walking, and provides a sense of security by conversing with them while they are standing up. Furthermore, the conversation support unit supports daily conversation with elderly persons, acting as a conversation partner to alleviate loneliness. The health monitoring unit monitors the health condition based on the conversation supported by the conversation support unit. For example, the health monitoring unit monitors the vital signs of elderly persons in real time, such as heart rate, blood pressure, and body temperature. The health monitoring unit also learns behavioral patterns of elderly persons, such as wake-up times and meal times. The abnormality detection unit detects abnormalities based on the health condition monitored by the health monitoring unit. For example, the abnormality detection unit detects abnormalities in heart rate or blood pressure and issues alerts when heart rate is abnormally high or low. The abnormality detection unit also detects abnormalities in behavioral patterns, such as not waking up at the usual time or remaining inactive for a long period, and issues alerts. The cooperation unit cooperates with a care taxi or medical system based on abnormalities detected by the abnormality detection unit. For example, the cooperation unit arranges a care taxi or makes emergency contact with a medical institution when an abnormality is detected. As a result, the care support system according to the embodiment can support the daily life and safety of elderly persons and assist in health management. Specifically, this care support system is composed of independent hardware and software modules for each unit, which are linked via a network to achieve advanced information processing beyond conventional simple automation. The operation support unit receives three-axis acceleration data (e.g., 100 samples per second, each sample containing x, y, z axis acceleration values) from wearable devices with built-in accelerometers and gyroscopes, and encoder values from walkers and robots (e.g., walking speed 0.5 m/s, stride length 0.4 m) as input, and determines movement states such as “walking,” “standing up,” and “sitting” using a deep learning-based movement recognition model (e.g., 1D convolutional neural network). The output consists of movement state labels (e.g., “walking,” “standing up”) and support intensity instruction values (e.g., 0.8=strong assistance), which are used to generate actuator control signals for walkers and robots. The conversation support unit converts voice input (e.g., 16 kHz sampled voice waveform data, content such as “The weather is nice today”) into text using a speech recognition model (e.g., transformer-based speech recognition network), and then generates conversation or reminders using a large language model. The output consists of text for speech synthesis and tone instructions for conversation content (e.g., “gentle,” “encouraging”). The health monitoring unit receives vital signs obtained from wearable sensors (e.g., heart rate 70 bpm, blood pressure 120/80 mmHg, body temperature 36.5° C. as time-series data) as input and sends them to the abnormality detection unit. The abnormality detection unit uses an LSTM-based time-series abnormality detection model to analyze input vital signs and behavioral patterns (e.g., delayed wake-up time, decreased activity) as multidimensional vectors and outputs abnormality scores (e.g., 0.85, exceeding the abnormality threshold of 0.7) and abnormality labels (e.g., “high risk,” “caution required”). The cooperation unit receives abnormality notifications from the abnormality detection unit and sends necessary information (e.g., elderly person ID, abnormality type, occurrence time, location information) to care taxi dispatch APIs and medical institution notification APIs, and issues push notifications to family members and medical professionals. Unlike conventional human observation or rule-based processing, these processes are autonomously performed by AI, which integrates multiple sensor information and extracts patterns in high-dimensional feature space, greatly improving accuracy, speed, and reproducibility. Technical effects include earlier abnormality detection, reduced false alarm rates, reduced burden on care sites, and realization of remote health management. Application fields include home care, care facilities, medical institutions, remote monitoring services, and rehabilitation support.

The operation support unit can provide walking assistance. For example, the operation support unit supports the walking of elderly persons using a walker, stabilizing their walking. The operation support unit can also support walking using a walking assistance robot, and can further support walking using a walking assistance device. For example, the operation support unit assists the walking of elderly persons using a walking assistance device. This supports the walking of elderly persons and reduces the risk of falls. Specifically, the operation support unit receives three-axis acceleration data (e.g., 100 samples per second, each sample containing x, y, z axis acceleration values) from wearable devices with built-in accelerometers and gyroscopes, and encoder values from walkers and robots (e.g., walking speed 0.5 m/s, stride length 0.4 m) as input. The operation support unit inputs these time-series data into a 1D convolutional neural network (CNN) or LSTM-type recurrent neural network to determine walking states. Examples of input include a 100-sample×3-axis acceleration tensor or a 10-second walking speed array. The output consists of movement state labels (e.g., “walking,” “stationary,” “high fall risk”) and support intensity instruction values (e.g., 0.8=strong assistance, 0.3=mild assistance). These output values are sent to actuator control signal generation modules for walkers and robots and are reflected in real-time assistance force and speed control. For AI model training, supervised learning using fall cases and normal walking data is applied, with cross-entropy or MSE as the loss function. Data augmentation includes noise addition and time-series shifting of walking patterns. Unlike conventional human observation or simple threshold judgment, the operation support unit autonomously extracts patterns in high-dimensional feature space and integrates multiple sensor information using AI, greatly improving the accuracy and reproducibility of walking state determination and fall prediction. Technical effects include early detection of abnormal walking, reduction of fall risk, reduction of caregiver burden, and realization of remote walking state monitoring. Application fields include home care, care facilities, rehabilitation support, and remote monitoring services.

The operation support unit can provide standing-up support. For example, the operation support unit supports the standing-up of elderly persons using a standing-up assistance device, stabilizing their standing-up. The operation support unit can also support standing-up using a standing-up support robot, and can further support standing-up using a standing-up aid. For example, the operation support unit assists the standing-up of elderly persons using a standing-up aid. This supports the standing-up of elderly persons and reduces the risk of falls. Specifically, the operation support unit receives acceleration sensor data and pressure sensor data during standing-up movements (e.g., seat pressure distribution, changes in foot pressure, 50 samples per second) as input. The operation support unit inputs these multidimensional time-series data into a 1D convolutional neural network or LSTM-type network to determine states such as “standing-up start,” “standing-up in progress,” and “standing-up complete.” Examples of input include a 50-sample ×4-channel tensor (left/right foot pressure+seat pressure+acceleration) or continuous waveform data of standing-up movements. The output consists of standing-up state labels (e.g., “standing-up in progress,” “complete”) and assistance intensity instruction values (e.g., 1.0=full assistance, 0.5=partial assistance), which are used to generate actuator control signals for assistance devices and robots. For AI model training, supervised learning using failed standing-up cases and normal movement data is applied, with cross-entropy or MSE as the loss function. Data augmentation includes variations in movement speed and addition of sensor noise. Unlike conventional human observation or simple threshold judgment, the operation support unit autonomously extracts patterns in high-dimensional feature space and integrates multiple sensor information using AI, greatly improving the accuracy and reproducibility of standing-up movement determination and fall prediction. Technical effects include early detection of standing-up failures, reduction of fall risk, reduction of caregiver burden, and realization of remote standing-up state monitoring. Application fields include home care, care facilities, rehabilitation support, and remote monitoring services.

The health monitoring unit can monitor vital signs in real time. For example, the health monitoring unit monitors vital signs such as heart rate, blood pressure, and body temperature in real time, detecting abnormalities in heart rate, blood pressure, and body temperature. This enables constant understanding of the health condition of elderly persons and early detection of abnormalities. Specifically, the health monitoring unit receives time-series array data of heart rate (e.g., 60-120 bpm, updated every minute), blood pressure (e.g., 100-180 mmHg, updated every 5 minutes), and body temperature (e.g., 35-39° C., updated every 10 minutes) obtained from wearable sensors as input. The health monitoring unit inputs these multidimensional time-series data into an LSTM-type time-series abnormality detection network or autoregressive model and outputs abnormality scores (e.g., 0.85, exceeding the abnormality threshold of 0.7) and abnormality labels (e.g., “high risk,” “caution required”). Examples of input include a 24-hour heart rate vector (1,440 elements), blood pressure vector (288 elements), and body temperature vector (144 elements). The output consists of abnormality scores and abnormality labels, which are sent to the abnormality detection unit and cooperation unit and used as triggers for alert issuance and medical institution notification. For AI model training, supervised learning and self-supervised learning using time-series data of normal and abnormal cases are applied, with binary cross-entropy or MSE as the loss function. Data augmentation includes noise addition and time-series shifting. Unlike conventional human observation or simple threshold judgment, the health monitoring unit autonomously extracts patterns in high-dimensional feature space and integrates multiple vital sign information using AI, greatly improving the accuracy and early detection capability of abnormality detection. Technical effects include earlier abnormality detection, reduced false alarm rates, reduced burden on care sites, and realization of remote health condition monitoring. Application fields include home care, care facilities, medical institutions, and remote monitoring services.

The health monitoring unit can learn behavioral patterns. For example, the health monitoring unit learns behavioral patterns such as wake-up times and meal times of elderly persons, and issues alerts when an elderly person does not wake up at the usual time or does not eat at the usual meal time. The health monitoring unit can also detect abnormal behavior, such as issuing alerts when an elderly person remains inactive for a long period. Specifically, the health monitoring unit receives behavioral data obtained from accelerometers and position sensors (e.g., activity level per minute, travel distance, time-series labels for sitting, standing, walking) as input. The health monitoring unit inputs these multidimensional time-series data into an LSTM-type behavioral pattern learning model or autoencoder to distinguish between normal and abnormal patterns. Examples of input include a 24-hour activity vector (1,440 elements), time-series arrays of wake-up and sleep times, and timestamp arrays of meal events. The output consists of abnormal behavior scores (e.g., 0.9, exceeding the threshold of 0.7) and abnormal behavior labels (e.g., “prolonged inactivity,” “delayed wake-up”), which are sent to the abnormality detection unit and cooperation unit and used as triggers for alert issuance and caregiver notification. For AI model training, supervised learning and clustering using time-series data of normal and abnormal behavioral patterns are applied, with cross-entropy or reconstruction error as the loss function. Data augmentation includes time-series shifting and noise addition for behavioral patterns. Unlike conventional human observation or simple rule-based processing, the health monitoring unit autonomously extracts patterns in high-dimensional feature space and integrates multiple sensor information using AI, greatly improving the accuracy and early detection capability of abnormal behavior detection. Technical effects include early detection of abnormal behavior, reduced false alarm rates, reduced burden on care sites, and realization of remote behavioral pattern monitoring. Application fields include home care, care facilities, medical institutions, and remote monitoring services.

The cooperation unit can analyze the condition of an elderly person using voice guidance. For example, the cooperation unit analyzes breathing sounds and coughing sounds of elderly persons using voice guidance, detecting signs of pneumonia and other abnormalities. The cooperation unit can also analyze speech content using voice guidance to detect signs of dementia. Specifically, the cooperation unit receives spectrogram data of breathing and coughing sounds (e.g., 1-second mel spectrogram, 128 dimensions×100 frames) and text data of speech content (e.g., “I've been forgetting things lately”) obtained from the voice analysis unit as input. The cooperation unit inputs these acoustic features into a CNN-based acoustic abnormality detection model and a transformer-based natural language processing model, and outputs abnormality probabilities (e.g., 0.92 abnormal, 0.08 normal), abnormality types (e.g., “wheezing,” “wet cough”), and dementia sign scores (e.g., 0.85, exceeding the threshold of 0.7). Examples of input include breathing sound spectrogram tensors (128×100), cough waveform data, and speech text sequences. The output consists of abnormality probabilities, abnormality types, and dementia sign scores, which are sent to care taxi dispatch APIs and medical institution notification APIs, and push notifications are sent to family members or medical professionals as needed. For AI model training, supervised learning using acoustic data of abnormal and normal sounds and dementia speech cases is applied, with cross-entropy or MSE as the loss function. Data augmentation includes noise addition and voice pitch conversion. Unlike conventional human auscultation or simple keyword detection, the cooperation unit autonomously extracts patterns in high-dimensional acoustic and natural language feature spaces using AI, greatly improving the accuracy and early detection capability of abnormal sign detection. Technical effects include early detection of abnormal signs, reduced false alarm rates, reduced burden on care sites, and realization of remote condition analysis. Application fields include home care, care facilities, medical institutions, and remote monitoring services.

The cooperation unit can perform necessary treatment and emergency contact. For example, the cooperation unit arranges a care taxi when an abnormality is detected, transporting the elderly person to a medical institution. The cooperation unit can also make emergency contact with medical institutions and family members when an abnormality is detected, reporting the situation. This enables rapid necessary treatment and emergency contact when an abnormality is detected in an elderly person. Specifically, the cooperation unit receives abnormality notification data (e.g., elderly person ID, abnormality type, occurrence time, location information, abnormality score) received from the abnormality detection unit and health monitoring unit as input. The cooperation unit generates and sends appropriate requests to care taxi dispatch APIs, medical institution notification APIs, and family notification APIs based on this structured data. Examples of input include abnormality type “heart rate abnormality,” occurrence time “2024-06-01 10:15,” location “home,” abnormality score 0.92. The output consists of API request data (e.g., JSON format dispatch request, emergency contact message) and notification result response data. These outputs are linked to subsequent care taxi dispatch systems, medical institution reception systems, and family smartphone apps. The AI model or rule engine automatically selects the cooperation destination and notification content according to the type and urgency of the abnormality. Unlike conventional manual phone contact or dispatch, the cooperation unit autonomously and rapidly processes from abnormality detection to cooperation, realizing faster emergency response and reduced human error. Technical effects include shortened response time in emergencies, reduced false alarm rates, reduced burden on care sites, and realization of remote emergency response. Application fields include home care, care facilities, medical institutions, and remote monitoring services.

A voice analysis unit is provided, and the voice analysis unit can analyze breathing sounds and coughing sounds of elderly persons. For example, the voice analysis unit analyzes breathing sounds to detect signs of pneumonia, using voice analysis algorithms to detect abnormalities in breathing sounds. The voice analysis unit can also analyze coughing sounds to detect abnormalities, using voice analysis algorithms to detect abnormalities in coughing sounds. Furthermore, the voice analysis unit can analyze breathing sounds and coughing sounds and detect abnormalities based on abnormal sound detection criteria. For example, the voice analysis unit detects abnormalities in breathing sounds and coughing sounds based on abnormal sound detection criteria. This enables early detection of signs such as pneumonia by analyzing breathing sounds and coughing sounds of elderly persons. Specifically, the voice analysis unit receives voice waveform data of breathing and coughing sounds of elderly persons obtained from microphones or wearable devices (e.g., 16 kHz sampling, 1-second 1D waveform, examples such as “wheeze,” “cough”) as input. The voice analysis unit performs preprocessing such as noise removal and normalization, and feature extraction such as mel spectrogram conversion (e.g., 128 dimensions×100 frames). The extracted acoustic features are then input into a convolutional neural network (CNN), recurrent neural network (RNN), or transformer-based acoustic abnormality detection model with self-attention mechanism. Examples of input include breathing sound spectrogram tensors (128×100), cough waveform data (16,000 samples per second). The AI model uses labeled datasets of normal and abnormal sounds (e.g., wheezing, wet cough, apnea) for supervised learning, with cross-entropy or mean squared error (MSE) as the loss function. The model outputs structured data such as abnormality probability (e.g., 0.92 abnormal, 0.08 normal), abnormality type (e.g., “wheezing,” “wet cough”), and abnormality score (e.g., 0.85, exceeding the threshold of 0.7). Specific output examples include “breathing sound abnormality probability 0.92, abnormality type: wheezing,” “cough sound abnormality probability 0.88, abnormality type: wet cough.” These output values are sent to subsequent abnormality detection units and cooperation units and used as triggers for threshold judgment and emergency contact. For AI model training, transfer learning and data augmentation (e.g., noise addition, voice pitch conversion, time stretching) are used to achieve high-accuracy abnormality detection even in small data environments. Unlike conventional human auscultation or simple threshold judgment, the voice analysis unit autonomously extracts patterns in high-dimensional acoustic feature space and integrates multiple acoustic sensor information using AI, greatly improving the accuracy, speed, and reproducibility of abnormal sound detection. Technical effects include early detection of signs of pneumonia and respiratory diseases, reduced false alarm rates, reduced burden on care sites, and realization of remote health condition monitoring. Application fields include home care, care facilities, medical institutions, remote monitoring services, and rehabilitation support.

The voice analysis unit can analyze the speech content of elderly persons. For example, the voice analysis unit analyzes speech content to detect signs of dementia, using speech recognition technology to detect abnormalities in speech content. The voice analysis unit can also use semantic analysis technology to detect abnormalities in speech content. Furthermore, the voice analysis unit can analyze speech content and detect abnormalities based on abnormal sound detection criteria. For example, the voice analysis unit detects abnormalities in speech content based on abnormal sound detection criteria. This enables early detection of signs such as dementia by analyzing the speech content of elderly persons. Specifically, the voice analysis unit receives speech audio data of elderly persons obtained from microphones or wearable devices (e.g., 16 kHz sampling, 10 seconds of audio waveform, examples such as “I've been forgetting things lately,” “What day is it today?”) as input. The voice analysis unit converts the audio waveform into text data using a speech recognition model (e.g., transformer-based speech recognition network or CTC-based speech recognition model). Examples of input include 10 seconds of audio waveform (160,000 samples), and output examples include text sequences such as “What day is it today?” The converted text data is then input into a natural language processing model (e.g., large language model, BERT, GPT series) for semantic analysis and abnormal speech detection. The AI model uses labeled datasets of normal speech and dementia sign speech (e.g., repeated questions, topic deviation, word confusion) for supervised learning, with cross-entropy or MSE as the loss function. The model outputs structured data such as abnormal speech probability (e.g., 0.87 abnormal, 0.13 normal), abnormality type (e.g., “repeated questions,” “incoherent speech”), and dementia sign score (e.g., 0.85, exceeding the threshold of 0.7). Specific output examples include “abnormal speech probability 0.87, abnormality type: repeated questions,” “dementia sign score 0.85.” These output values are sent to subsequent abnormality detection units and cooperation units and used as triggers for threshold judgment and medical institution notification. For AI model training, transfer learning and data augmentation (e.g., speech speed variation, noise addition, paraphrase generation) are used to achieve high-accuracy abnormality detection even in small data environments. Unlike conventional human conversation observation or simple keyword detection, the voice analysis unit autonomously extracts patterns in high-dimensional language feature space and integrates multiple speech history information using AI, greatly improving the accuracy, speed, and reproducibility of dementia sign detection. Technical effects include early detection of signs of dementia, reduced false alarm rates, reduced burden on care sites, and realization of remote cognitive function monitoring. Application fields include home care, care facilities, medical institutions, remote monitoring services, and rehabilitation support.

The operation support unit can estimate the emotion of an elderly person and adjust the timing of operation support based on the estimated emotion. For example, if the elderly person feels anxious, the operation support unit advances the timing of operation support to provide a sense of security. If the elderly person is relaxed, the operation support unit delays the timing to respect autonomy. If the elderly person is in a hurry, the operation support unit provides support quickly to facilitate smooth movement. By adjusting the timing of operation support according to the emotion of the elderly person, more appropriate support can be provided. Emotion estimation is realized using, for example, an emotion engine or generative AI with emotion estimation functions, such as text generation AI (e.g., LLM) or multimodal generative AI, but is not limited thereto. Specifically, the operation support unit receives voice data of elderly persons obtained from microphones or wearable devices (e.g., 16 kHz sampling, 10 seconds of audio waveform, examples such as “I'm a little anxious,” “I'm feeling good today”), facial image data (e.g., 640×480 pixels, 5 frames per second), and biometric sensor data (e.g., heart rate, skin potential, time-series vector per minute) as input. The operation support unit performs preprocessing such as noise removal, normalization, and face region extraction, and extracts voice features (e.g., mel-frequency cepstral coefficients), image features (e.g., facial landmark vectors), and biometric features (e.g., heart rate variability indices). These features are input into a multimodal emotion estimation model (e.g., transformer-based network integrating voice, image, and biometric signals), which outputs emotion labels (e.g., “anxiety,” “relaxation,” “hurry”) and emotion scores (e.g., anxiety level 0.82, relaxation level 0.15). Examples of input include 10 seconds of audio waveform (160,000 samples), 5-frame facial image tensor (5×640×480×3), and heart rate vector (10 elements). Output examples include “emotion label: anxiety, emotion score: 0.82,” “emotion label: relaxation, emotion score: 0.15.” These output values are sent to the operation support timing determination module, which automatically adjusts the timing of walking assistance or standing-up assistance based on rules such as “advance support timing by 2 seconds if anxiety level exceeds 0.7” or “delay support timing by 5 seconds if relaxation level exceeds 0.8.” For AI model training, supervised learning and transfer learning using emotion-labeled voice, image, and biometric datasets are applied, with cross-entropy or MSE as the loss function. Data augmentation includes voice noise addition, facial image rotation and brightness change, and time-series shifting of heart rate data. Unlike conventional subjective human observation or simple threshold judgment, the operation support unit autonomously extracts patterns in high-dimensional feature space and integrates multiple modality information using AI, greatly improving the accuracy, speed, and reproducibility of emotion estimation. Technical effects include optimal timing of operation support according to emotional state, maximization of security and autonomy, reduction of fall risk and stress, reduction of caregiver burden, and realization of remote emotion state monitoring. Application fields include home care, care facilities, medical institutions, remote monitoring services, rehabilitation support, and emotion-adaptive robot control.

The operation support unit can analyze the past operation history of an elderly person and select an optimal operation support method. For example, if the elderly person has a history of falls, the operation support unit strengthens support to prevent falls. If the elderly person has a history of needing walking assistance, the operation support unit increases the frequency of walking assistance. If the elderly person has a history of needing standing-up support, the operation support unit focuses on standing-up support. By analyzing the past operation history of elderly persons, optimal operation support methods can be provided. Specifically, the operation support unit receives operation history data of elderly persons accumulated in a time-series database (e.g., time-series array of walking state labels, timestamps of fall events, history of standing-up assistance, frequency of each event, support intensity instruction values) as input. The operation support unit inputs these history data into time-series analysis models such as LSTM-type recurrent neural networks or autoencoders to extract past operation patterns and trends of abnormal events. Examples of input include a one-year array of walking state labels (365 days×24 hours×60 minutes), timestamp array of fall events (e.g., 2023-12-01 09:15, 2024-01-10 14:30), and standing-up assistance history vector (e.g., number of assistance per day). The AI model outputs fall risk scores (e.g., 0.92, exceeding the threshold of 0.7), walking assistance necessity scores (e.g., 0.85), standing-up assistance necessity scores (e.g., 0.78), support policy labels such as “high fall risk, increased walking assistance recommended,” “focus on standing-up assistance recommended,” and specific support intensity instruction values (e.g., walking assistance intensity 0.9, standing-up assistance intensity 0.8). These output values are sent to the operation support control module and used for actuator control signal generation and assistance timing determination for walkers and robots. For AI model training, supervised learning and clustering using normal and abnormal operation history (fall and assistance request events) are applied, with cross-entropy or reconstruction error as the loss function. Data augmentation includes time-series shifting and noise addition of history data and generation of event frequency variations. Unlike conventional human reference to history or simple rule-based processing, the operation support unit autonomously extracts patterns in high-dimensional history feature space and integrates multiple history information using AI, greatly improving the accuracy and reproducibility of support policy optimization. Technical effects include early prediction of fall risk, optimal allocation of support resources, reduction of caregiver burden, and realization of remote history-based operation support. Application fields include home care, care facilities, rehabilitation support, remote monitoring services, and personalized robot control.

The operation support unit can customize the support content during operation support based on the current physical condition and environment of an elderly person. For example, if the elderly person is in poor physical condition, the operation support unit reduces the intensity of support. If the elderly person is in a hot environment, the operation support unit increases the frequency of support to prevent exhaustion. If the elderly person is in a cold environment, the operation support unit adjusts the timing of support to prevent a drop in body temperature. By customizing the support content according to the current physical condition and environment of elderly persons, more appropriate support can be provided. Specifically, the operation support unit receives vital signs obtained from wearable sensors (e.g., time-series vectors of heart rate, blood pressure, body temperature), environmental data obtained from environmental sensors (e.g., temperature, humidity, atmospheric pressure; room temperature 28° C., humidity 70%, pressure 1010 hPa), and subjective physical condition reports from elderly persons (e.g., text input such as “I'm not feeling well today”) as input. The operation support unit performs preprocessing such as normalization, outlier removal, and feature extraction (e.g., heart rate variability indices, temperature/humidity change rates), and inputs the data into a multimodal environment-adaptive support determination model (e.g., transformer-based network integrating vital, environmental, and subjective information). Examples of input include a one-minute heart rate vector (60 elements), time-series arrays of room temperature and humidity (24 hours), and physical condition report text sequences (e.g., “It's hot today,” “I feel chilly”). The AI model outputs support intensity instruction values (e.g., 0.3=mild assistance, 0.8=strong assistance), support frequency instruction values (e.g., 1.5 times increase), and support timing adjustment values (e.g., 5 minutes earlier than usual). Output examples include “support intensity 0.3, frequency 1.5 times, timing −5 minutes.” These output values are sent to the operation support control module and reflected in actuator control and assistance scheduling for walkers and robots. For AI model training, supervised learning and transfer learning using datasets of physical condition, environment, subjective information, and support policy correspondence are applied, with cross-entropy or MSE as the loss function. Data augmentation includes noise addition to environmental data and paraphrase generation for physical condition reports. Unlike conventional subjective human judgment or simple threshold judgment, the operation support unit autonomously extracts patterns in high-dimensional environment and physical condition feature space and integrates multiple information using AI, greatly improving the accuracy and reproducibility of support content optimization. Technical effects include improved responsiveness to changes in physical condition and environment, prevention of excessive or insufficient support, reduction of caregiver burden, and realization of remote environment-adaptive support. Application fields include home care, care facilities, rehabilitation support, remote monitoring services, and personalized robot control.

The operation support unit can estimate the emotion of an elderly person and determine the priority of operation support based on the estimated emotion. For example, if the elderly person feels anxious, the operation support unit sets the priority of operation support high. If the elderly person is relaxed, the operation support unit sets the priority low. If the elderly person is in a hurry, the operation support unit sets the priority to the highest. By determining the priority of operation support according to the emotion of the elderly person, more appropriate support can be provided. Emotion estimation is realized using, for example, an emotion engine or generative AI with emotion estimation functions, such as text generation AI (e.g., LLM) or multimodal generative AI, but is not limited thereto. Specifically, the operation support unit receives voice data of elderly persons obtained from microphones or wearable devices (e.g., 16 kHz sampling, 10 seconds of audio waveform, examples such as “I'm a little anxious,” “I'm feeling good today”), facial image data (e.g., 640×480 pixels, 5 frames per second), and biometric sensor data (e.g., heart rate, skin potential, time-series vector per minute) as input. The operation support unit performs preprocessing such as noise removal, normalization, and face region extraction, and extracts voice features (e.g., mel-frequency cepstral coefficients), image features (e.g., facial landmark vectors), and biometric features (e.g., heart rate variability indices). These features are input into a multimodal emotion estimation model (e.g., transformer-based network integrating voice, image, and biometric signals), which outputs emotion labels (e.g., “anxiety,” “relaxation,” “hurry”) and emotion scores (e.g., anxiety level 0.82, relaxation level 0.15). Examples of input include 10 seconds of audio waveform (160,000 samples), 5-frame facial image tensor (5×640×480×3), and heart rate vector (10 elements). Output examples include “emotion label: anxiety, emotion score: 0.82,” “emotion label: relaxation, emotion score: 0.15.” These output values are sent to the operation support priority determination module, which automatically adjusts the priority of walking assistance or standing-up assistance based on rules such as “set priority to highest if anxiety level exceeds 0.7” or “set priority low if relaxation level exceeds 0.8.” For AI model training, supervised learning and transfer learning using emotion-labeled voice, image, and biometric datasets are applied, with cross-entropy or MSE as the loss function. Data augmentation includes voice noise addition, facial image rotation and brightness change, and time-series shifting of heart rate data. Unlike conventional subjective human observation or simple threshold judgment, the operation support unit autonomously extracts patterns in high-dimensional feature space and integrates multiple modality information using AI, greatly improving the accuracy, speed, and reproducibility of emotion estimation and priority determination. Technical effects include optimal priority of operation support according to emotional state, maximization of security and autonomy, reduction of fall risk and stress, reduction of caregiver burden, and realization of remote emotion-adaptive support. Application fields include home care, care facilities, medical institutions, remote monitoring services, and emotion-adaptive robot control.

The operation support unit can select an optimal support method during operation support by considering the geographic location information of an elderly person. For example, if the elderly person is at home, the operation support unit focuses on indoor operation support. If the elderly person is outside, the operation support unit focuses on walking assistance and standing-up support. If the elderly person is in a facility, the operation support unit focuses on operation support within the facility. By providing optimal operation support methods based on the geographic location information of elderly persons, more appropriate support can be provided. Specifically, the operation support unit receives geographic location information of elderly persons obtained from GPS modules, Wi-Fi/Bluetooth beacons, and indoor location estimation sensors (e.g., latitude and longitude coordinates, labels for home/facility/outside, room numbers) as input. The operation support unit records these location information as time-series data and inputs them into a geographic context estimation model (e.g., LSTM-type network for location label sequences or decision tree model for geographic features). Examples of input include a 24-hour location label array (e.g., “home,” “outside,” “facility”), indoor coordinate vectors (e.g., x=12.5, y=8.3), and room numbers (e.g., room 201). The AI model outputs support method labels (e.g., “indoor walking assistance,” “outdoor walking assistance,” “standing-up assistance”) and support intensity instruction values (e.g., 0.7=indoor assistance, 0.9=outdoor assistance). Output examples include “support method: indoor walking assistance, intensity 0.7,” “support method: outdoor walking assistance, intensity 0.9.” These output values are sent to the operation support control module and used for actuator control and automatic switching of assistance content for walkers and robots. For AI model training, supervised learning and clustering using datasets of location information and support policy correspondence are applied, with cross-entropy or MSE as the loss function. Data augmentation includes time-series shifting and noise addition for location labels. Unlike conventional human location confirmation or simple rule-based processing, the operation support unit autonomously extracts patterns in high-dimensional location feature space and integrates multiple location information using AI, greatly improving the accuracy and reproducibility of support method selection. Technical effects include optimal support according to geographic situation, prevention of falls and accidents, reduction of caregiver burden, and realization of remote location-adaptive support. Application fields include home care, care facilities, rehabilitation support, remote monitoring services, and location-adaptive robot control.

The operation support unit can analyze the social media activity of an elderly person during operation support and provide relevant support. For example, if the elderly person shows interest in exercise on social media, the operation support unit focuses on exercise support. If the elderly person shows interest in health on social media, the operation support unit focuses on health management support. If the elderly person shows interest in hobbies on social media, the operation support unit provides support related to those hobbies. By providing relevant operation support based on the social media activity of elderly persons, more appropriate support can be provided. Specifically, the operation support unit obtains social media post data of elderly persons (e.g., text posts, image posts, like history, list of followed accounts) via API and inputs them into natural language processing models (e.g., BERT or large language models) and image feature extraction models (e.g., CNN-based image classification networks). Examples of input include text posts from the past month (e.g., “I recently started walking,” “I'm paying attention to my health”), image posts (e.g., photos of exercising), and like history (e.g., reactions to health-related posts). The AI model outputs interest area labels (e.g., “exercise,” “health,” “hobby: gardening”) and interest scores (e.g., exercise 0.85, health 0.78, gardening 0.65). Output examples include “interest area: exercise, interest score 0.85,” “interest area: health, interest score 0.78.” These output values are sent to the operation support policy determination module, which automatically adjusts support content based on rules such as “focus on exercise support if exercise interest score exceeds 0.8” or “strengthen health management support if health interest score exceeds 0.7.” For AI model training, supervised learning and clustering using datasets of social media activity and support policy correspondence are applied, with cross-entropy or MSE as the loss function. Data augmentation includes paraphrase generation for post text and rotation/brightness change for image data. Unlike conventional human post confirmation or simple keyword extraction, the operation support unit autonomously extracts patterns in high-dimensional social feature space and integrates multiple post information using AI, greatly improving the accuracy, speed, and reproducibility of interest area estimation and support content optimization. Technical effects include personalized support according to individual interests, reduction of caregiver burden, and realization of remote interest-adaptive support. Application fields include home care, care facilities, rehabilitation support, remote monitoring services, and personalized robot control.

The conversation support unit can estimate the emotion of an elderly person and adjust the content and tone of conversation based on the estimated emotion. For example, if the elderly person is sad, the conversation support unit conducts conversation with comforting content and tone. If the elderly person is happy, the conversation support unit conducts conversation with empathetic content and tone. If the elderly person is angry, the conversation support unit conducts conversation with calming content and tone. By adjusting the content and tone of conversation according to the emotion of the elderly person, more appropriate conversation support can be provided. Emotion estimation is realized using, for example, an emotion engine or generative AI with emotion estimation functions, such as text generation AI (e.g., LLM) or multimodal generative AI, but is not limited thereto. Specifically, the conversation support unit receives voice data of elderly persons obtained from microphones or wearable devices (e.g., 16 kHz sampling, 10 seconds of audio waveform, examples such as “I'm lonely today,” “I had a happy event”), facial image data (e.g., 640×480 pixels, 5 frames per second), and biometric sensor data (e.g., heart rate, skin potential, time-series vector per minute) as input. The conversation support unit performs preprocessing such as noise removal, normalization, and face region extraction, and extracts voice features (e.g., mel-frequency cepstral coefficients), image features (e.g., facial landmark vectors), and biometric features (e.g., heart rate variability indices). These features are input into a multimodal emotion estimation model (e.g., transformer-based network integrating voice, image, and biometric signals), which outputs emotion labels (e.g., “sadness,” “joy,” “anger”) and emotion scores (e.g., sadness level 0.82, joy level 0.15). Examples of input include 10 seconds of audio waveform (160,000 samples), 5-frame facial image tensor (5×640×480×3), and heart rate vector (10 elements). Output examples include “emotion label: sadness, emotion score: 0.82,” “emotion label: joy, emotion score: 0.15.” These output values are sent to the conversation generation module, which automatically adjusts conversation content and tone based on rules such as “generate comforting content and tone if sadness level exceeds 0.7” or “generate empathetic content and tone if joy level exceeds 0.8.” For AI model training, supervised learning and transfer learning using emotion-labeled voice, image, and biometric datasets are applied, with cross-entropy or MSE as the loss function. Data augmentation includes voice noise addition, facial image rotation and brightness change, and time-series shifting of heart rate data. Unlike conventional subjective human observation or simple threshold judgment, the conversation support unit autonomously extracts patterns in high-dimensional feature space and integrates multiple modality information using AI, greatly improving the accuracy, speed, and reproducibility of emotion estimation and conversation content/tone adjustment. Technical effects include optimal conversation support according to emotional state, maximization of security and empathy, reduction of loneliness and stress, reduction of caregiver burden, and realization of remote emotion-adaptive conversation support. Application fields include home care, care facilities, medical institutions, remote monitoring services, and emotion-adaptive robot conversation.

The conversation support unit can refer to the past conversation history of an elderly person during conversation support and provide optimal conversation content. For example, the conversation support unit provides relevant topics based on content previously discussed by the elderly person, provides conversation content based on topics previously of interest, and adjusts conversation content based on topics previously avoided. By referring to the past conversation history of elderly persons, optimal conversation content can be provided. Specifically, the conversation support unit receives conversation history data of elderly persons accumulated in a time-series database (e.g., conversation text sequences from the past year, time-series of topic labels, conversation frequency, and emotion scores during conversation) as input. The conversation support unit inputs these history data into time-series analysis models such as LSTM-type recurrent neural networks or autoencoders to extract past conversation patterns and trends of interest and avoided topics. Examples of input include a 365-day conversation text array (e.g., “I went for a walk yesterday,” “I want to avoid talking about travel”), topic label array (e.g., “health,” “hobby,” “family”), and emotion score vector during conversation (e.g., joy 0.7, sadness 0.2). The AI model outputs recommended topic labels (e.g., “health,” “hobby”), avoided topic labels (e.g., “travel”), and context vectors for conversation content generation. Output examples include “recommended topic: health,” “avoided topic: travel,” “conversation content generation context: hobby/gardening.” These output values are sent to the conversation generation module, which automatically generates optimal conversation content based on past conversation history. For AI model training, supervised learning and clustering using datasets of conversation history and topic selection/avoidance history are applied, with cross-entropy or reconstruction error as the loss function. Data augmentation includes paraphrase generation for conversation text and time-series shifting for topic labels. Unlike conventional human reference to history or simple rule-based processing, the conversation support unit autonomously extracts patterns in high-dimensional history feature space and integrates multiple history information using AI, greatly improving the accuracy and reproducibility of conversation content optimization. Technical effects include personalized conversation according to individual preferences and history, improved conversation satisfaction, reduction of stress and discomfort, reduction of caregiver burden, and realization of remote history-adaptive conversation support. Application fields include home care, care facilities, rehabilitation support, remote monitoring services, and personalized robot conversation.

The conversation support unit can customize the conversation content during conversation support based on the current interests and hobbies of an elderly person. For example, the conversation support unit provides topics related to hobbies currently of interest to the elderly person, topics related to news or events currently of interest, and topics related to projects currently being undertaken. By customizing the conversation content according to the current interests and hobbies of elderly persons, more appropriate conversation support can be provided. Specifically, the conversation support unit receives data related to the current interests and hobbies of elderly persons (e.g., conversation content from the past month, hobby/interest labels, news browsing history, project progress notes) as input. The conversation support unit inputs these data into natural language processing models (e.g., large language models or BERT-based models) and clustering algorithms to extract current interest areas and hobby trends. Examples of input include recent conversation text (e.g., “I've been into gardening lately,” “I watched a soccer match”), hobby labels (e.g., “gardening,” “sports viewing”), and news browsing history (e.g., health-related news, local event information). The AI model outputs recommended topic labels (e.g., “gardening,” “soccer”), interest scores (e.g., gardening 0.85, soccer 0.78), and context vectors for conversation generation. Output examples include “recommended topic: gardening, interest score 0.85,” “recommended topic: soccer, interest score 0.78.” These output values are sent to the conversation generation module, which automatically generates conversation content based on current interests and hobbies. For AI model training, supervised learning and clustering using datasets of interests/hobbies and conversation content correspondence are applied, with cross-entropy or reconstruction error as the loss function. Data augmentation includes paraphrase generation for hobby labels and time-series shifting for news data. Unlike conventional human confirmation of interests or simple keyword extraction, the conversation support unit autonomously extracts patterns in high-dimensional interest feature space and integrates multiple information using AI, greatly improving the accuracy and reproducibility of conversation content customization. Technical effects include personalized conversation according to individual interests, improved conversation satisfaction, reduction of caregiver burden, and realization of remote interest-adaptive conversation support. Application fields include home care, care facilities, rehabilitation support, remote monitoring services, and personalized robot conversation.

The conversation support unit can estimate the emotion of an elderly person and adjust the frequency of conversation based on the estimated emotion. For example, if the elderly person is feeling lonely, the conversation support unit increases the frequency of conversation to alleviate the sense of loneliness. For instance, when the elderly person feels lonely, the conversation support unit increases the frequency of conversation to reduce loneliness. Additionally, if the elderly person is feeling stressed, the conversation support unit can decrease the frequency of conversation to help the person relax. For example, when the elderly person feels stressed, the conversation support unit decreases the frequency of conversation to promote relaxation. Furthermore, if the elderly person is enjoying themselves, the conversation support unit can maintain the frequency of conversation at an appropriate level. For example, when the elderly person is enjoying themselves, the conversation support unit keeps the conversation frequency moderate. By adjusting the frequency of conversation according to the emotion of the elderly person, more appropriate conversation support can be provided. Emotion estimation is realized using, for example, an emotion engine or a generative AI with emotion estimation functionality. The generative AI may be a text generation AI (e.g., LLM) or a multimodal generative AI, but is not limited to these examples. Specifically, the conversation support unit receives as input multimodal data obtained from microphones or wearable devices, such as voice data of the elderly person (e.g., 16 kHz sampling, 10-second audio waveform, content examples such as “I feel lonely,” “Today is fun”), facial image data (e.g., 640×480 pixel face images, 5 frames per second), and biometric sensor data (e.g., heart rate, skin potential, time-series vectors every minute). The conversation support unit performs preprocessing on these multimodal data, such as noise removal, normalization, and face region extraction, and extracts audio features (e.g., Mel-frequency cepstral coefficients), image features (e.g., facial expression landmark vectors), and biometric features (e.g., heart rate variability indices). The extracted features are then input to a multimodal emotion estimation model (e.g., a Transformer-type network integrating audio, image, and biometric signals), which outputs emotion labels (e.g., “loneliness,” “stress,” “enjoyment”) and emotion scores (e.g., loneliness score 0.82, enjoyment score 0.15). Examples of input include a 10-second audio waveform (160,000 samples), a face image tensor of 5 frames (5×640×480×3), and a heart rate vector (10 elements). Examples of output include “Emotion label: loneliness, emotion score: 0.82” and “Emotion label: enjoyment, emotion score: 0.15.” These output values are sent to the conversation frequency determination module, which automatically adjusts the conversation frequency based on rules such as “If the loneliness score exceeds 0.7, increase the conversation frequency by 1.5 times,” “If the stress score exceeds 0.8, decrease the conversation frequency by 0.5 times,” and so on. For AI model training, supervised learning or transfer learning is applied using datasets of audio, image, and biometric data labeled with emotions, and loss functions such as cross-entropy or MSE are used. Data augmentation includes adding noise to audio, rotating or changing the brightness of facial images, and time-series shifting of heart rate data. Unlike conventional subjective human observation or simple threshold judgment, the conversation support unit autonomously performs pattern extraction in high-dimensional feature space and integrated analysis of multiple modalities using AI, greatly improving the accuracy, speed, and reproducibility of emotion estimation and conversation frequency adjustment. Technical effects include optimal conversation frequency provision according to emotional state, reduction of loneliness and stress, maximization of comfort and enjoyment, reduction of burden in care settings, and realization of emotion-adaptive remote conversation support. Application fields include home care, care facilities, medical institutions, remote monitoring services, and emotion-adaptive robot conversation.

The conversation support unit can provide relevant topics during conversation support by considering the geographic location information of the elderly person. For example, if the elderly person is at home, the conversation support unit provides topics related to the home area. For instance, when the elderly person is at home, the conversation support unit provides topics about the surrounding area. Additionally, if the elderly person is outside, the conversation support unit can provide topics related to the location outside. For example, when the elderly person is outside, the conversation support unit provides topics about the place they are visiting. Furthermore, if the elderly person is in a facility, the conversation support unit can provide topics related to the facility. For example, when the elderly person is in a facility, the conversation support unit provides topics about the facility. By providing relevant topics based on the geographic location information of the elderly person, more appropriate conversation support can be provided. Specifically, the conversation support unit receives as input the geographic location information of the elderly person obtained from GPS modules, Wi-Fi/Bluetooth beacons, indoor location estimation sensors, etc. (e.g., latitude and longitude coordinates, labels such as home, facility, or outside, room numbers, etc.). The conversation support unit records these location data as a time series and inputs them into a geographic context estimation model (e.g., an LSTM-type network using location label sequences as input or a decision tree model using geographic features as input). Examples of input include a 24-hour location label array (e.g., “home,” “outside,” “facility”), indoor coordinate vectors (e.g., x=12.5, y=8.3), and room numbers (e.g., room 201). The AI model outputs recommended topic labels (e.g., “home area news,” “outside event,” “facility activity”) and context vectors for topic generation. Examples of output include “Recommended topic: home area news” and “Recommended topic: outside event.” These output values are sent to the conversation generation module, and relevant topics based on geographic location information are automatically generated. For AI model training, supervised learning or clustering is applied using datasets of location information and topic selection correspondence, and loss functions such as cross-entropy or MSE are used. Data augmentation includes time-series shifting of location labels and adding noise. Unlike conventional human location confirmation or simple rule-based processing, the conversation support unit autonomously performs pattern extraction in high-dimensional location feature space and integrated analysis of multiple location information using AI, greatly improving the accuracy and reproducibility of topic selection. Technical effects include optimal topic provision according to geographic situation, improved conversation satisfaction, reduced burden in care settings, and realization of location-adaptive remote conversation support. Application fields include home care, care facilities, rehabilitation support, remote monitoring services, and location-adaptive robot conversation.

The conversation support unit can analyze the social media activity of the elderly person during conversation support and provide relevant topics. For example, if the elderly person shows interest in certain topics on social media, the conversation support unit provides those topics. For instance, when the elderly person shows interest in topics on social media, the conversation support unit provides those topics. Additionally, the conversation support unit can provide topics related to people followed by the elderly person on social media. For example, when the elderly person follows certain people on social media, the conversation support unit provides topics about those people. Furthermore, the conversation support unit can provide topics related to content shared by the elderly person on social media. For example, when the elderly person shares content on social media, the conversation support unit provides topics about that content. By providing relevant topics based on the social media activity of the elderly person, more appropriate conversation support can be provided. Specifically, the conversation support unit obtains the elderly person's social media post data (e.g., text posts, image posts, like history, list of followed accounts, etc.) via API and inputs them into a natural language processing model (e.g., BERT or large language models) and an image feature extraction model (e.g., CNN-based image classification network). Examples of input include text posts from the past month (e.g., “I recently started walking,” “I'm paying attention to my health”), image posts (e.g., photos during exercise), like history (e.g., reactions to health-related posts), and followed accounts (e.g., celebrities, friends). The AI model outputs recommended topic labels (e.g., “exercise,” “health,” “hobby: gardening”), interest scores (e.g., exercise 0.85, health 0.78, gardening 0.65), and topics about followed people (e.g., “new post by XX”). Examples of output include “Recommended topic: exercise, interest score 0.85,” “Recommended topic: health, interest score 0.78,” “Topic about followed person: friend's travelogue,” etc. These output values are sent to the conversation generation module, and relevant topics based on social media activity are automatically generated. For AI model training, supervised learning or clustering is applied using datasets of social media activity and topic selection correspondence, and loss functions such as cross-entropy or MSE are used. Data augmentation includes paraphrase generation of post texts and rotation/brightness changes of image data. Unlike conventional human post confirmation or simple keyword extraction, the conversation support unit autonomously performs pattern extraction in high-dimensional social feature space and integrated analysis of multiple post information using AI, greatly improving the accuracy, speed, and reproducibility of topic estimation and conversation content optimization. Technical effects include personalized conversation provision according to individual interests, improved conversation satisfaction, reduced burden in care settings, and realization of interest-adaptive remote conversation support. Application fields include home care, care facilities, rehabilitation support, remote monitoring services, and personalized robot conversation.

The health monitoring unit can estimate the emotion of an elderly person and adjust the frequency of health monitoring based on the estimated emotion. For example, if the elderly person feels anxious, the health monitoring unit increases the frequency of health monitoring to provide a sense of security. For instance, when the elderly person feels anxious, the health monitoring unit increases the frequency of health monitoring to provide reassurance. Additionally, if the elderly person is relaxed, the health monitoring unit can decrease the frequency of health monitoring to respect autonomy. For example, when the elderly person is relaxed, the health monitoring unit decreases the frequency of health monitoring to respect autonomy. Furthermore, if the elderly person is in a hurry, the health monitoring unit can perform health monitoring more quickly to support smooth health management. For example, when the elderly person is in a hurry, the health monitoring unit performs health monitoring rapidly to support smooth health management. By adjusting the frequency of health monitoring according to the emotion of the elderly person, more appropriate health management can be provided. Emotion estimation is realized using, for example, an emotion engine or a generative AI with emotion estimation functionality. The generative AI may be a text generation AI (e.g., LLM) or a multimodal generative AI, but is not limited to these examples. Specifically, the health monitoring unit receives as input multimodal data obtained from microphones or wearable devices, such as voice data of the elderly person (e.g., 16 kHz sampling, 10-second audio waveform, content examples such as “I feel anxious today,” “I am calm”), facial image data (e.g., 640×480 pixel face images, 5 frames per second), and biometric sensor data (e.g., heart rate, skin potential, time-series vectors every minute). The health monitoring unit performs preprocessing on these multimodal data, such as noise removal, normalization, and face region extraction, and extracts audio features (e.g., Mel-frequency cepstral coefficients), image features (e.g., facial expression landmark vectors), and biometric features (e.g., heart rate variability indices). The extracted features are then input to a multimodal emotion estimation model (e.g., a Transformer-type network integrating audio, image, and biometric signals), which outputs emotion labels (e.g., “anxiety,” “relaxation,” “hurry”) and emotion scores (e.g., anxiety score 0.82, relaxation score 0.15). Examples of input include a 10-second audio waveform (160,000 samples), a face image tensor of 5 frames (5×640×480×3), and a heart rate vector (10 elements). Examples of output include “Emotion label: anxiety, emotion score: 0.82” and “Emotion label: relaxation, emotion score: 0.15.” These output values are sent to the health monitoring frequency determination module, which automatically adjusts the health monitoring frequency based on rules such as “If the anxiety score exceeds 0.7, increase the monitoring frequency by 1.5 times,” “If the relaxation score exceeds 0.8, decrease the monitoring frequency by 0.5 times,” and so on. For AI model training, supervised learning or transfer learning is applied using datasets of audio, image, and biometric data labeled with emotions, and loss functions such as cross-entropy or MSE are used. Data augmentation includes adding noise to audio, rotating or changing the brightness of facial images, and time-series shifting of heart rate data. Unlike conventional subjective human observation or simple threshold judgment, the health monitoring unit autonomously performs pattern extraction in high-dimensional feature space and integrated analysis of multiple modalities using AI, greatly improving the accuracy, speed, and reproducibility of emotion estimation and monitoring frequency adjustment. Technical effects include optimal health monitoring frequency provision according to emotional state, maximization of reassurance and autonomy, early detection of abnormalities, reduction of burden in care settings, and realization of emotion-adaptive remote health monitoring. Application fields include home care, care facilities, medical institutions, remote monitoring services, and emotion-adaptive health management.

The health monitoring unit can refer to the past health data of the elderly person during health monitoring to perform early detection of abnormalities. For example, if the elderly person has a history of abnormal heart rate, the health monitoring unit strengthens heart rate monitoring. For instance, when the elderly person has a history of abnormal heart rate, the health monitoring unit strengthens heart rate monitoring. Additionally, if the elderly person has a history of abnormal blood pressure, the health monitoring unit can strengthen blood pressure monitoring. For example, when the elderly person has a history of abnormal blood pressure, the health monitoring unit strengthens blood pressure monitoring. Furthermore, if the elderly person has a history of abnormal body temperature, the health monitoring unit can strengthen body temperature monitoring. For example, when the elderly person has a history of abnormal body temperature, the health monitoring unit strengthens body temperature monitoring. By referring to the past health data of the elderly person, early detection of abnormalities can be achieved. Specifically, the health monitoring unit receives as input the health history data of the elderly person accumulated in a time-series database (e.g., heart rate vectors for the past year, blood pressure vectors, body temperature vectors, timestamps of abnormal events, frequency of abnormal occurrences, etc.). The health monitoring unit inputs these history data into time-series analysis models such as LSTM-type recurrent neural networks or autoencoders to extract past abnormal patterns and occurrence trends. Examples of input include heart rate vectors for 365 days (365×24×60 elements), blood pressure vectors (365×288 elements), body temperature vectors (365×144 elements), and abnormal event timestamp arrays (e.g., 2023-12-01 09:15, 2024-01-10 14:30). The AI model outputs abnormal occurrence risk scores (e.g., 0.92, exceeding threshold 0.7), recommended labels for enhanced monitoring (e.g., “enhanced heart rate monitoring,” “enhanced blood pressure monitoring”). Examples of output include “High risk of heart rate abnormality, increased monitoring frequency recommended,” “High risk of blood pressure abnormality, enhanced monitoring recommended,” etc. These output values are sent to the health monitoring control module and used for automatic adjustment of monitoring frequency and threshold settings for heart rate, blood pressure, and body temperature. For AI model training, supervised learning or clustering is applied using normal and abnormal history (abnormal event occurrences, etc.), and loss functions such as cross-entropy or reconstruction error are used. Data augmentation includes time-series shifting of history data, adding noise, and generating variations in abnormal event frequency. Unlike conventional human history reference or simple rule-based processing, the health monitoring unit autonomously performs pattern extraction in high-dimensional history feature space and integrated analysis of multiple history information using AI, greatly improving the speed and reproducibility of early abnormality detection. Technical effects include early prediction of abnormal risk, optimal allocation of monitoring resources, reduction of burden in care settings, and realization of history-based remote health monitoring. Application fields include home care, care facilities, medical institutions, remote monitoring services, and personalized health management.

The health monitoring unit can customize the monitoring content during health monitoring based on the current living conditions of the elderly person. For example, if the elderly person is feeling unwell, the health monitoring unit reduces the intensity of health monitoring. For instance, when the elderly person is feeling unwell, the health monitoring unit reduces the intensity of health monitoring. Additionally, if the elderly person is in a hot environment, the health monitoring unit can increase the frequency of health monitoring to prevent physical exhaustion. For example, when the elderly person is in a hot environment, the health monitoring unit increases the frequency of health monitoring to prevent physical exhaustion. Furthermore, if the elderly person is in a cold environment, the health monitoring unit can adjust the timing of health monitoring to prevent a drop in body temperature. For example, when the elderly person is in a cold environment, the health monitoring unit adjusts the timing of health monitoring to prevent a decrease in body temperature. By customizing the monitoring content based on the current living conditions of the elderly person, more appropriate health management can be provided. Specifically, the health monitoring unit receives as input vital signs obtained from wearable sensors (e.g., time-series vectors of heart rate, blood pressure, body temperature), environmental sensor data (e.g., temperature, humidity, atmospheric pressure; e.g., room temperature 28° C., humidity 70%, pressure 1010 hPa), and subjective health status reports from the elderly person (e.g., text input such as “I am not feeling well today”). The health monitoring unit performs preprocessing on these multidimensional data, such as normalization, outlier removal, and feature extraction (e.g., heart rate variability indices, temperature/humidity change rates), and inputs them into a multimodal environment-adaptive monitoring decision model (e.g., a Transformer-type network integrating vital, environmental, and subjective information). Examples of input include a heart rate vector every minute (60 elements), time-series arrays of room temperature and humidity (24 hours), and health status report text sequences (e.g., “It's hot today,” “I feel chilly”). The AI model outputs monitoring intensity instruction values (e.g., 0.3=mild monitoring, 0.8=strong monitoring), monitoring frequency instruction values (e.g., 1.5 times increase), and monitoring timing adjustment values (e.g., 5 minutes earlier than usual). Examples of output include specific control parameters such as “monitoring intensity 0.3, frequency 1.5 times, timing −5 minutes.” These output values are sent to the health monitoring control module and used for automatic adjustment of monitoring content according to vital signs and environmental data. For AI model training, supervised learning or transfer learning is applied using datasets of health status, environment, subjective information, and monitoring policies, and loss functions such as cross-entropy or MSE are used. Data augmentation includes adding noise to environmental data and generating paraphrases of health status reports. Unlike conventional subjective human judgment or simple threshold judgment, the health monitoring unit autonomously performs pattern extraction in high-dimensional environment/health feature space and integrated analysis of multiple information using AI, greatly improving the accuracy and reproducibility of monitoring content optimization. Technical effects include improved responsiveness to health and environmental changes, prevention of excessive or insufficient monitoring, reduction of burden in care settings, and realization of environment-adaptive remote health monitoring. Application fields include home care, care facilities, rehabilitation support, remote monitoring services, and personalized health management.

The health monitoring unit can estimate the emotion of an elderly person and determine the priority of health monitoring based on the estimated emotion. For example, if the elderly person feels anxious, the health monitoring unit sets the priority of health monitoring high. For instance, when the elderly person feels anxious, the health monitoring unit sets the priority of health monitoring high. Additionally, if the elderly person is relaxed, the health monitoring unit can set the priority of health monitoring low. For example, when the elderly person is relaxed, the health monitoring unit sets the priority of health monitoring low. Furthermore, if the elderly person is in a hurry, the health monitoring unit can set the priority of health monitoring to the highest. For example, when the elderly person is in a hurry, the health monitoring unit sets the priority of health monitoring to the highest. By determining the priority of health monitoring according to the emotion of the elderly person, more appropriate health management can be provided. Emotion estimation is realized using, for example, an emotion engine or a generative AI with emotion estimation functionality. The generative AI may be a text generation AI (e.g., LLM) or a multimodal generative AI, but is not limited to these examples. Specifically, the health monitoring unit receives as input multimodal data obtained from microphones or wearable devices, such as voice data of the elderly person (e.g., 16 kHz sampling, 10-second audio waveform, content examples such as “I feel anxious today,” “I am calm”), facial image data (e.g., 640×480 pixel face images, 5 frames per second), and biometric sensor data (e.g., heart rate, skin potential, time-series vectors every minute). The health monitoring unit performs preprocessing on these multimodal data, such as noise removal, normalization, and face region extraction, and extracts audio features (e.g., Mel-frequency cepstral coefficients), image features (e.g., facial expression landmark vectors), and biometric features (e.g., heart rate variability indices). The extracted features are then input to a multimodal emotion estimation model (e.g., a Transformer-type network integrating audio, image, and biometric signals), which outputs emotion labels (e.g., “anxiety,” “relaxation,” “hurry”) and emotion scores (e.g., anxiety score 0.82, relaxation score 0.15). Examples of input include a 10-second audio waveform (160,000 samples), a face image tensor of 5 frames (5×640×480×3), and a heart rate vector (10 elements). Examples of output include “Emotion label: anxiety, emotion score: 0.82” and “Emotion label: relaxation, emotion score: 0.15.” These output values are sent to the health monitoring priority determination module, which automatically adjusts the priority of health monitoring based on rules such as “If the anxiety score exceeds 0.7, set the monitoring priority to the highest,” “If the relaxation score exceeds 0.8, set the monitoring priority low,” and so on. For AI model training, supervised learning or transfer learning is applied using datasets of audio, image, and biometric data labeled with emotions, and loss functions such as cross-entropy or MSE are used. Data augmentation includes adding noise to audio, rotating or changing the brightness of facial images, and time-series shifting of heart rate data. Unlike conventional subjective human observation or simple threshold judgment, the health monitoring unit autonomously performs pattern extraction in high-dimensional feature space and integrated analysis of multiple modalities using AI, greatly improving the accuracy, speed, and reproducibility of emotion estimation and priority determination. Technical effects include optimal health monitoring priority provision according to emotional state, maximization of reassurance and autonomy, early detection of abnormalities, reduction of burden in care settings, and realization of emotion-adaptive remote health monitoring. Application fields include home care, care facilities, medical institutions, remote monitoring services, and emotion-adaptive health management.

The health monitoring unit can select the optimal monitoring method during health monitoring by considering the geographic location information of the elderly person. For example, if the elderly person is at home, the health monitoring unit focuses on health monitoring around the home. For instance, when the elderly person is at home, the health monitoring unit focuses on health monitoring around the home. Additionally, if the elderly person is outside, the health monitoring unit can focus on health monitoring at the location outside. For example, when the elderly person is outside, the health monitoring unit focuses on health monitoring at the location outside. Furthermore, if the elderly person is in a facility, the health monitoring unit can focus on health monitoring within the facility. For example, when the elderly person is in a facility, the health monitoring unit focuses on health monitoring within the facility. By providing the optimal health monitoring method based on the geographic location information of the elderly person, more appropriate health monitoring can be provided. Specifically, the health monitoring unit receives as input the geographic location information of the elderly person obtained from GPS modules, Wi-Fi/Bluetooth beacons, indoor location estimation sensors, etc. (e.g., latitude and longitude coordinates, labels such as home, facility, or outside, room numbers, etc.). The health monitoring unit records these location data as a time series and inputs them into a geographic context estimation model (e.g., an LSTM-type network using location label sequences as input or a decision tree model using geographic features as input). Examples of input include a 24-hour location label array (e.g., “home,” “outside,” “facility”), indoor coordinate vectors (e.g., x=12.5, y=8.3), and room numbers (e.g., room 201). The AI model outputs monitoring method labels (e.g., “home area monitoring,” “outside monitoring,” “facility monitoring”) and monitoring intensity instruction values (e.g., 0.7=home monitoring, 0.9=outside monitoring). Examples of output include “Monitoring method: home area monitoring, intensity 0.7” and “Monitoring method: outside monitoring, intensity 0.9.” These output values are sent to the health monitoring control module and used for automatic switching of monitoring content and frequency for vital signs and behavioral patterns. For AI model training, supervised learning or clustering is applied using datasets of location information and monitoring policy correspondence, and loss functions such as cross-entropy or MSE are used. Data augmentation includes time-series shifting of location labels and adding noise. Unlike conventional human location confirmation or simple rule-based processing, the health monitoring unit autonomously performs pattern extraction in high-dimensional location feature space and integrated analysis of multiple location information using AI, greatly improving the accuracy and reproducibility of monitoring method selection. Technical effects include optimal health monitoring provision according to geographic situation, prevention of fall risk and accidents, reduction of burden in care settings, and realization of location-adaptive remote health monitoring. Application fields include home care, care facilities, rehabilitation support, remote monitoring services, and location-adaptive health management.

The health monitoring unit can analyze the social media activity of the elderly person during health monitoring and provide relevant health information. For example, if the elderly person shows interest in health on social media, the health monitoring unit provides relevant health information. For instance, when the elderly person shows interest in health on social media, the health monitoring unit provides relevant health information. Additionally, if the elderly person shows interest in exercise on social media, the health monitoring unit can provide health information related to exercise. For example, when the elderly person shows interest in exercise on social media, the health monitoring unit provides health information related to exercise. Furthermore, if the elderly person shows interest in diet on social media, the health monitoring unit can provide health information related to diet. For example, when the elderly person shows interest in diet on social media, the health monitoring unit provides health information related to diet. By providing relevant health information based on the social media activity of the elderly person, more appropriate health management can be provided. Specifically, the health monitoring unit obtains the elderly person's social media post data (e.g., text posts, image posts, like history, list of followed accounts, etc.) via API and inputs them into a natural language processing model (e.g., BERT or large language models) and an image feature extraction model (e.g., CNN-based image classification network). Examples of input include text posts from the past month (e.g., “I recently started walking,” “I'm paying attention to my health”), image posts (e.g., photos during exercise), like history (e.g., reactions to health-related posts), and followed accounts (e.g., celebrities, friends). The AI model outputs interest area labels (e.g., “exercise,” “health,” “diet”), interest scores (e.g., exercise 0.85, health 0.78, diet 0.65). Examples of output include “Interest area: exercise, interest score 0.85,” “Interest area: health, interest score 0.78,” “Interest area: diet, interest score 0.65,” etc. These output values are sent to the health information recommendation module, which automatically adjusts the content and presentation frequency of health information based on rules such as “If exercise interest score exceeds 0.8, focus on providing health information related to exercise,” “If diet interest score exceeds 0.7, strengthen health information related to diet,” and so on. For AI model training, supervised learning or clustering is applied using datasets of social media activity and health information recommendation correspondence, and loss functions such as cross-entropy or MSE are used. Data augmentation includes paraphrase generation of post texts and rotation/brightness changes of image data. Unlike conventional human post confirmation or simple keyword extraction, the health monitoring unit autonomously performs pattern extraction in high-dimensional social feature space and integrated analysis of multiple post information using AI, greatly improving the accuracy, speed, and reproducibility of interest area estimation and health information optimization. Technical effects include personalized health information provision according to individual interests, reduction of burden in care settings, and realization of interest-adaptive remote health management. Application fields include home care, care facilities, rehabilitation support, remote monitoring services, and personalized health management.

The abnormality detection unit can estimate the emotion of an elderly person and adjust the criteria for abnormality detection based on the estimated emotion. For example, if the elderly person feels anxious, the abnormality detection unit sets stricter criteria for abnormality detection. For instance, when the elderly person feels anxious, the abnormality detection unit sets stricter criteria for abnormality detection. Additionally, if the elderly person is relaxed, the abnormality detection unit can set looser criteria for abnormality detection. For example, when the elderly person is relaxed, the abnormality detection unit sets looser criteria for abnormality detection. Furthermore, if the elderly person is in a hurry, the abnormality detection unit can set the criteria for abnormality detection more quickly. For example, when the elderly person is in a hurry, the abnormality detection unit sets the criteria for abnormality detection more quickly. By adjusting the criteria for abnormality detection according to the emotion of the elderly person, more appropriate abnormality detection can be provided. Emotion estimation is realized using, for example, an emotion engine or a generative AI with emotion estimation functionality. The generative AI may be a text generation AI (e.g., LLM) or a multimodal generative AI, but is not limited to these examples. Specifically, the abnormality detection unit receives as input multimodal data obtained from microphones or wearable devices, such as voice data of the elderly person (e.g., 16 kHz sampling, 10-second audio waveform, content examples such as “I feel anxious today,” “I am calm”), facial image data (e.g., 640×480 pixel face images, 5 frames per second), and biometric sensor data (e.g., heart rate, skin potential, time-series vectors every minute). The abnormality detection unit performs preprocessing on these multimodal data, such as noise removal, normalization, and face region extraction, and extracts audio features (e.g., Mel-frequency cepstral coefficients), image features (e.g., facial expression landmark vectors), and biometric features (e.g., heart rate variability indices). The extracted features are then input to a multimodal emotion estimation model (e.g., a Transformer-type network integrating audio, image, and biometric signals), which outputs emotion labels (e.g., “anxiety,” “relaxation,” “hurry”) and emotion scores (e.g., anxiety score 0.82, relaxation score 0.15). Examples of input include a 10-second audio waveform (160,000 samples), a face image tensor of 5 frames (5×640×480×3), and a heart rate vector (10 elements). Examples of output include “Emotion label: anxiety, emotion score: 0.82” and “Emotion label: relaxation, emotion score: 0.15.” The abnormality detection unit sends these emotion estimation results to the abnormality detection criteria adjustment module, which automatically adjusts the abnormality detection thresholds and algorithm parameters for heart rate, blood pressure, body temperature, behavioral patterns, etc., based on rules such as “If anxiety score exceeds 0.7, decrease the abnormality detection threshold by 0.1,” “If relaxation score exceeds 0.8, increase the abnormality detection threshold by 0.1,” and so on. For AI model training, supervised learning or transfer learning is applied using datasets of audio, image, and biometric data labeled with emotions and abnormality detection criteria adjustment history, and loss functions such as cross-entropy or MSE are used. Data augmentation includes adding noise to audio, rotating or changing the brightness of facial images, and time-series shifting of heart rate data. Unlike conventional subjective human observation or simple threshold judgment, the abnormality detection unit autonomously performs pattern extraction in high-dimensional feature space and integrated analysis of multiple modalities using AI, realizing dynamic optimization of abnormality detection criteria according to emotional state, and greatly improving the accuracy, speed, and reproducibility of abnormality detection. Technical effects include optimal abnormality detection criteria provision according to emotional state, reduction of false alarm rate, maximization of reassurance and autonomy, early detection of abnormalities, reduction of burden in care settings, and realization of emotion-adaptive remote abnormality detection. Application fields include home care, care facilities, medical institutions, remote monitoring services, and emotion-adaptive health management.

The abnormality detection unit can refer to the past abnormality data of the elderly person during abnormality detection to perform early detection of abnormalities. For example, if the elderly person has a history of abnormal heart rate, the abnormality detection unit detects abnormal heart rate early. For instance, when the elderly person has a history of abnormal heart rate, the abnormality detection unit detects abnormal heart rate early. Additionally, if the elderly person has a history of abnormal blood pressure, the abnormality detection unit can detect abnormal blood pressure early. For example, when the elderly person has a history of abnormal blood pressure, the abnormality detection unit detects abnormal blood pressure early. Furthermore, if the elderly person has a history of abnormal body temperature, the abnormality detection unit can detect abnormal body temperature early. For example, when the elderly person has a history of abnormal body temperature, the abnormality detection unit detects abnormal body temperature early. By referring to the past abnormality data of the elderly person, early detection of abnormalities can be achieved. Specifically, the abnormality detection unit receives as input the abnormality history data of the elderly person accumulated in a time-series database (e.g., timestamps of heart rate abnormality events for the past year, blood pressure abnormality history, body temperature abnormality history, time-series vectors of abnormality frequency and abnormality scores). The abnormality detection unit inputs these history data into time-series analysis models such as LSTM-type recurrent neural networks or autoencoders to extract past abnormality occurrence patterns and trends. Examples of input include heart rate abnormality event arrays for 365 days (e.g., 2023-12-01 09:15, 2024-01-10 14:30), blood pressure abnormality history vectors (365×288 elements), body temperature abnormality history vectors (365×144 elements). The AI model outputs abnormality occurrence risk scores (e.g., 0.92, exceeding threshold 0.7), predicted timing of abnormality occurrence (e.g., next occurrence prediction “2024-07-01 08:00”), and recommended labels for enhanced monitoring (e.g., “enhanced heart rate monitoring,” “enhanced blood pressure monitoring”). Examples of output include “High risk of heart rate abnormality, increased monitoring frequency recommended,” “High risk of blood pressure abnormality, enhanced monitoring recommended,” “Predicted body temperature abnormality: tomorrow at 8 a.m. ,” etc. These output values are sent to the abnormality detection control module and used for automatic adjustment of monitoring frequency and threshold settings for heart rate, blood pressure, and body temperature, as well as for issuing abnormality prediction alerts. For AI model training, supervised learning or clustering is applied using normal and abnormal history (abnormal event occurrences, etc.), and loss functions such as cross-entropy or reconstruction error are used. Data augmentation includes time-series shifting of history data, adding noise, and generating variations in abnormal event frequency. Unlike conventional human history reference or simple rule-based processing, the abnormality detection unit autonomously performs pattern extraction in high-dimensional history feature space and integrated analysis of multiple history information using AI, greatly improving the speed and reproducibility of early abnormality detection. Technical effects include early prediction of abnormality risk, optimal allocation of monitoring resources, reduction of burden in care settings, and realization of history-based remote abnormality detection. Application fields include home care, care facilities, medical institutions, remote monitoring services, and personalized health management.

The abnormality detection unit can customize the detection content during abnormality detection based on the current living conditions of the elderly person. For example, if the elderly person is feeling unwell, the abnormality detection unit relaxes the criteria for abnormality detection. For instance, when the elderly person is feeling unwell, the abnormality detection unit relaxes the criteria for abnormality detection. Additionally, if the elderly person is in a hot environment, the abnormality detection unit can increase the frequency of abnormality detection to prevent physical exhaustion. For example, when the elderly person is in a hot environment, the abnormality detection unit increases the frequency of abnormality detection to prevent physical exhaustion. Furthermore, if the elderly person is in a cold environment, the abnormality detection unit can adjust the timing of abnormality detection to prevent a drop in body temperature. For example, when the elderly person is in a cold environment, the abnormality detection unit adjusts the timing of abnormality detection to prevent a decrease in body temperature. By customizing the detection content based on the current living conditions of the elderly person, more appropriate abnormality detection can be provided. Specifically, the abnormality detection unit receives as input vital signs obtained from wearable sensors (e.g., time-series vectors of heart rate, blood pressure, body temperature), environmental sensor data (e.g., temperature, humidity, atmospheric pressure; e.g., room temperature 28° C., humidity 70%, pressure 1010 hPa), and subjective health status reports from the elderly person (e.g., text input such as “I am not feeling well today”). The abnormality detection unit performs preprocessing on these multidimensional data, such as normalization, outlier removal, and feature extraction (e.g., heart rate variability indices, temperature/humidity change rates), and inputs them into a multimodal environment-adaptive abnormality detection model (e.g., a Transformer-type network integrating vital, environmental, and subjective information). Examples of input include a heart rate vector every minute (60 elements), time-series arrays of room temperature and humidity (24 hours), and health status report text sequences (e.g., “It's hot today,” “I feel chilly”). The AI model outputs abnormality detection intensity instruction values (e.g., 0.3=mild detection, 0.8=strong detection), abnormality detection frequency instruction values (e.g., 1.5 times increase), and abnormality detection timing adjustment values (e.g., 5 minutes earlier than usual). Examples of output include specific control parameters such as “detection intensity 0.3, frequency 1.5 times, timing −5 minutes.” These output values are sent to the abnormality detection control module and used for automatic adjustment of abnormality detection content according to vital signs and environmental data. For AI model training, supervised learning or transfer learning is applied using datasets of health status, environment, subjective information, and abnormality detection policies, and loss functions such as cross-entropy or MSE are used. Data augmentation includes adding noise to environmental data and generating paraphrases of health status reports. Unlike conventional subjective human judgment or simple threshold judgment, the abnormality detection unit autonomously performs pattern extraction in high-dimensional environment/health feature space and integrated analysis of multiple information using AI, greatly improving the accuracy and reproducibility of abnormality detection content optimization. Technical effects include improved responsiveness to health and environmental changes, prevention of excessive or insufficient abnormality detection, reduction of burden in care settings, and realization of environment-adaptive remote abnormality detection. Application fields include home care, care facilities, rehabilitation support, remote monitoring services, and personalized health management.

The abnormality detection unit can estimate the emotion of an elderly person and determine the priority of abnormality detection based on the estimated emotion. For example, if the elderly person feels anxious, the abnormality detection unit sets the priority of abnormality detection high. For instance, when the elderly person feels anxious, the abnormality detection unit sets the priority of abnormality detection high. Additionally, if the elderly person is relaxed, the abnormality detection unit can set the priority of abnormality detection low. For example, when the elderly person is relaxed, the abnormality detection unit sets the priority of abnormality detection low. Furthermore, if the elderly person is in a hurry, the abnormality detection unit can set the priority of abnormality detection to the highest. For example, when the elderly person is in a hurry, the abnormality detection unit sets the priority of abnormality detection to the highest. By determining the priority of abnormality detection according to the emotion of the elderly person, more appropriate abnormality detection can be provided. Emotion estimation is realized using, for example, an emotion engine or a generative AI with emotion estimation functionality. The generative AI may be a text generation AI (e.g., LLM) or a multimodal generative AI, but is not limited to these examples. Specifically, the abnormality detection unit receives as input multimodal data obtained from microphones or wearable devices, such as voice data of the elderly person (e.g., 16 kHz sampling, 10-second audio waveform, content examples such as “I feel anxious today,” “I am calm”), facial image data (e.g., 640×480 pixel face images, 5 frames per second), and biometric sensor data (e.g., heart rate, skin potential, time-series vectors every minute). The abnormality detection unit performs preprocessing on these multimodal data, such as noise removal, normalization, and face region extraction, and extracts audio features (e.g., Mel-frequency cepstral coefficients), image features (e.g., facial expression landmark vectors), and biometric features (e.g., heart rate variability indices). The extracted features are then input to a multimodal emotion estimation model (e.g., a Transformer-type network integrating audio, image, and biometric signals), which outputs emotion labels (e.g., “anxiety,” “relaxation,” “hurry”) and emotion scores (e.g., anxiety score 0.82, relaxation score 0.15). Examples of input include a 10-second audio waveform (160,000 samples), a face image tensor of 5 frames (5×640×480×3), and a heart rate vector (10 elements). Examples of output include “Emotion label: anxiety, emotion score: 0.82” and “Emotion label: relaxation, emotion score: 0.15.” The abnormality detection unit sends these emotion estimation results to the abnormality detection priority determination module, which automatically adjusts the priority of abnormality detection processing for heart rate, blood pressure, body temperature, behavioral patterns, etc., based on rules such as “If anxiety score exceeds 0.7, set the abnormality detection priority to the highest,” “If relaxation score exceeds 0.8, set the priority low,” and so on. For AI model training, supervised learning or transfer learning is applied using datasets of audio, image, and biometric data labeled with emotions and abnormality detection priority adjustment history, and loss functions such as cross-entropy or MSE are used. Data augmentation includes adding noise to audio, rotating or changing the brightness of facial images, and time-series shifting of heart rate data. Unlike conventional subjective human observation or simple threshold judgment, the abnormality detection unit autonomously performs pattern extraction in high-dimensional feature space and integrated analysis of multiple modalities using AI, realizing dynamic optimization of abnormality detection priority according to emotional state, and greatly improving the accuracy, speed, and reproducibility of abnormality detection. Technical effects include optimal abnormality detection priority provision according to emotional state, reduction of false alarm rate, maximization of reassurance and autonomy, early detection of abnormalities, reduction of burden in care settings, and realization of emotion-adaptive remote abnormality detection. Application fields include home care, care facilities, medical institutions, remote monitoring services, and emotion-adaptive health management.

The abnormality detection unit can select the optimal detection method during abnormality detection by considering the geographic location information of the elderly person. For example, if the elderly person is at home, the abnormality detection unit focuses on abnormality detection around the home. For instance, when the elderly person is at home, the abnormality detection unit focuses on abnormality detection around the home. Additionally, if the elderly person is outside, the abnormality detection unit can focus on abnormality detection at the location outside. For example, when the elderly person is outside, the abnormality detection unit focuses on abnormality detection at the location outside. Furthermore, if the elderly person is in a facility, the abnormality detection unit can focus on abnormality detection within the facility. For example, when the elderly person is in a facility, the abnormality detection unit focuses on abnormality detection within the facility. By providing the optimal abnormality detection method based on the geographic location information of the elderly person, more appropriate abnormality detection can be provided. Specifically, the abnormality detection unit receives as input the geographic location information of the elderly person obtained from GPS modules, Wi-Fi/Bluetooth beacons, indoor location estimation sensors, etc. (e.g., latitude and longitude coordinates, labels such as home, facility, or outside, room numbers, etc.). The abnormality detection unit records these location data as a time series and inputs them into a geographic context estimation model (e.g., an LSTM-type network using location label sequences as input or a decision tree model using geographic features as input). Examples of input include a 24-hour location label array (e.g., “home,” “outside,” “facility”), indoor coordinate vectors (e.g., x=12.5, y=8.3), and room numbers (e.g., room 201). The AI model outputs abnormality detection method labels (e.g., “home area detection,” “outside detection,” “facility detection”) and abnormality detection intensity instruction values (e.g., 0.7=home detection, 0.9=outside detection). Examples of output include “Detection method: home area detection, intensity 0.7” and “Detection method: outside detection, intensity 0.9.” These output values are sent to the abnormality detection control module and used for automatic switching of abnormality detection content and frequency for vital signs and behavioral patterns. For AI model training, supervised learning or clustering is applied using datasets of location information and abnormality detection policy correspondence, and loss functions such as cross-entropy or MSE are used. Data augmentation includes time-series shifting of location labels and adding noise. Unlike conventional human location confirmation or simple rule-based processing, the abnormality detection unit autonomously performs pattern extraction in high-dimensional location feature space and integrated analysis of multiple location information using AI, greatly improving the accuracy and reproducibility of detection method selection. Technical effects include optimal abnormality detection provision according to geographic situation, prevention of fall risk and accidents, reduction of burden in care settings, and realization of location-adaptive remote abnormality detection. Application fields include home care, care facilities, rehabilitation support, remote monitoring services, and location-adaptive health management.

The abnormality detection unit can analyze the social media activity of the elderly person during abnormality detection and provide relevant abnormality information. For example, if the elderly person shows interest in health on social media, the abnormality detection unit provides relevant abnormality information. For instance, when the elderly person shows interest in health on social media, the abnormality detection unit provides relevant abnormality information. Additionally, if the elderly person shows interest in exercise on social media, the abnormality detection unit can provide abnormality information related to exercise. For example, when the elderly person shows interest in exercise on social media, the abnormality detection unit provides abnormality information related to exercise. Furthermore, if the elderly person shows interest in diet on social media, the abnormality detection unit can provide abnormality information related to diet. For example, when the elderly person shows interest in diet on social media, the abnormality detection unit provides abnormality information related to diet. By providing relevant abnormality information based on the social media activity of the elderly person, more appropriate abnormality detection can be provided. Specifically, the abnormality detection unit obtains the elderly person's social media post data (e.g., text posts, image posts, like history, list of followed accounts, etc.) via API and inputs them into a natural language processing model (e.g., BERT or large language models) and an image feature extraction model (e.g., CNN-based image classification network). Examples of input include text posts from the past month (e.g., “I recently started walking,” “I'm paying attention to my health”), image posts (e.g., photos during exercise), like history (e.g., reactions to health-related posts), and followed accounts (e.g., celebrities, friends). The AI model outputs interest area labels (e.g., “exercise,” “health,” “diet”), interest scores (e.g., exercise 0.85, health 0.78, diet 0.65). Examples of output include “Interest area: exercise, interest score 0.85,” “Interest area: health, interest score 0.78,” “Interest area: diet, interest score 0.65,” etc. The abnormality detection unit sends these output values to the abnormality information recommendation module, which automatically adjusts the content and presentation frequency of abnormality information based on rules such as “If exercise interest score exceeds 0.8, focus on providing abnormality information related to exercise (e.g., fall risk during exercise, abnormal heart rate due to excessive exercise),” “If diet interest score exceeds 0.7, strengthen abnormality information related to diet (e.g., nutritional balance abnormality, abnormal food intake),” and so on. For AI model training, supervised learning or clustering is applied using datasets of social media activity and abnormality information recommendation correspondence, and loss functions such as cross-entropy or MSE are used. Data augmentation includes paraphrase generation of post texts and rotation/brightness changes of image data. Unlike conventional human post confirmation or simple keyword extraction, the abnormality detection unit autonomously performs pattern extraction in high-dimensional social feature space and integrated analysis of multiple post information using AI, greatly improving the accuracy, speed, and reproducibility of interest area estimation and abnormality information optimization. Technical effects include personalized abnormality information provision according to individual interests, reduction of burden in care settings, and realization of interest-adaptive remote abnormality detection. Application fields include home care, care facilities, rehabilitation support, remote monitoring services, and personalized health management.

The cooperation unit can estimate the emotion of an elderly person and adjust the timing of cooperation based on the estimated emotion. For example, if the elderly person feels anxious, the cooperation unit advances the timing of cooperation to provide reassurance. For instance, when the elderly person feels anxious, the cooperation unit advances the timing of cooperation to provide reassurance. Additionally, if the elderly person is relaxed, the cooperation unit can delay the timing of cooperation to respect autonomy. For example, when the elderly person is relaxed, the cooperation unit delays the timing of cooperation to respect autonomy. Furthermore, if the elderly person is in a hurry, the cooperation unit can perform cooperation quickly to support smooth cooperation. For example, when the elderly person is in a hurry, the cooperation unit performs cooperation quickly to support smooth cooperation. By adjusting the timing of cooperation according to the emotion of the elderly person, more appropriate cooperation can be provided. Emotion estimation is realized using, for example, an emotion engine or a generative AI with emotion estimation functionality. The generative AI may be a text generation AI (e.g., LLM) or a multimodal generative AI, but is not limited to these examples. Specifically, the cooperation unit receives as input multimodal data obtained from microphones or wearable devices, such as voice data of the elderly person (e.g., 16 kHz sampling, 10-second audio waveform, content examples such as “I feel a little anxious,” “I am calm today”), facial image data (e.g., 640×480 pixel face images, 5 frames per second), and biometric sensor data (e.g., heart rate, skin potential, time-series vectors every minute). The cooperation unit performs preprocessing on these multimodal data, such as noise removal, normalization, and face region extraction, and extracts audio features (e.g., Mel-frequency cepstral coefficients), image features (e.g., facial expression landmark vectors), and biometric features (e.g., heart rate variability indices). The extracted features are then input to a multimodal emotion estimation model (e.g., a Transformer-type network integrating audio, image, and biometric signals), which outputs emotion labels (e.g., “anxiety,” “relaxation,” “hurry”) and emotion scores (e.g., anxiety score 0.82, relaxation score 0.15). Examples of input include a 10-second audio waveform (160,000 samples), a face image tensor of 5 frames (5×640×480×3), and a heart rate vector (10 elements). Examples of output include “Emotion label: anxiety, emotion score: 0.82” and “Emotion label: relaxation, emotion score: 0.15.” The cooperation unit sends these emotion estimation results to the cooperation timing determination module, which automatically adjusts the timing of cooperation notifications or actions with care staff, medical institutions, or family members based on rules such as “If anxiety score exceeds 0.7, advance the cooperation timing by 10 minutes,” “If relaxation score exceeds 0.8, delay the cooperation timing by 15 minutes,” and so on. For AI model training, supervised learning or transfer learning is applied using datasets of audio, image, and biometric data labeled with emotions and cooperation timing adjustment history, and loss functions such as cross-entropy or MSE are used. Data augmentation includes adding noise to audio, rotating or changing the brightness of facial images, and time-series shifting of heart rate data. Unlike conventional subjective human observation or simple threshold judgment, the cooperation unit autonomously performs pattern extraction in high-dimensional feature space and integrated analysis of multiple modalities using AI, realizing dynamic optimization of cooperation timing according to emotional state, and greatly improving the accuracy, speed, and reproducibility of cooperation. Technical effects include optimal cooperation timing provision according to emotional state, maximization of reassurance and autonomy, rapid response in case of abnormality, reduction of burden in care settings, and realization of emotion-adaptive remote cooperation. Application fields include home care, care facilities, medical institutions, remote monitoring services, and emotion-adaptive cooperation support.

The cooperation unit can refer to the past cooperation history of the elderly person during cooperation to select the optimal cooperation method. For example, the cooperation unit proposes the optimal cooperation method based on the cooperation methods previously used by the elderly person. For instance, the cooperation unit proposes the optimal cooperation method based on the cooperation methods previously used by the elderly person. Additionally, the cooperation unit can propose the most efficient cooperation method based on the past cooperation history of the elderly person. For example, the cooperation unit proposes the most efficient cooperation method based on the past cooperation history of the elderly person. Furthermore, the cooperation unit can analyze the past cooperation history of the elderly person and propose the optimal cooperation timing. For example, the cooperation unit analyzes the past cooperation history of the elderly person and proposes the optimal cooperation timing. By referring to the past cooperation history of the elderly person, the optimal cooperation method can be provided. Specifically, the cooperation unit receives as input the cooperation history data of the elderly person accumulated in a time-series database (e.g., timestamps of cooperation events for the past year, cooperation method labels, types of cooperation partners, cooperation success rates, emotion scores at the time of cooperation, etc.). The cooperation unit inputs these history data into time-series analysis models such as LSTM-type recurrent neural networks or autoencoders to extract past cooperation patterns and trends in optimal cooperation methods and timing. Examples of input include cooperation event arrays for 365 days (e.g., 2023-12-01 09:15 “medical institution cooperation” success, 2024-01-10 14:30 “family cooperation” failure), cooperation method label arrays (e.g., “phone,” “app notification,” “visit”), and emotion score vectors at the time of cooperation (e.g., reassurance 0.7, anxiety 0.2). The AI model outputs recommended cooperation method labels (e.g., “phone,” “app notification”), recommended cooperation timing (e.g., 8 a.m., 6 p.m.), and cooperation success probability scores (e.g., 0.92). Examples of output include “Recommended cooperation method: phone, success probability 0.92,” “Recommended cooperation timing: 8 a.m. ,” etc. These output values are sent to the cooperation control module, and the optimal cooperation method and timing based on past history are automatically selected and executed. For AI model training, supervised learning or clustering is applied using datasets of cooperation history and cooperation results, and loss functions such as cross-entropy or reconstruction error are used. Data augmentation includes time-series shifting of cooperation events and generation of variations in cooperation method labels. Unlike conventional human history reference or simple rule-based processing, the cooperation unit autonomously performs pattern extraction in high-dimensional history feature space and integrated analysis of multiple history information using AI, greatly improving the accuracy and reproducibility of cooperation method selection and timing optimization. Technical effects include automatic proposal of optimal cooperation methods and timing based on past cooperation achievements, reduction of cooperation failure rate, reduction of burden in care settings, and realization of history-adaptive remote cooperation support. Application fields include home care, care facilities, medical institutions, remote monitoring services, and personalized cooperation support.

The cooperation unit can customize the cooperation content during cooperation based on the current living conditions of the elderly person. For example, if the elderly person is feeling unwell, the cooperation unit reduces the cooperation content. For instance, when the elderly person is feeling unwell, the cooperation unit reduces the cooperation content. Additionally, if the elderly person is in a hot environment, the cooperation unit can increase the frequency of cooperation to prevent physical exhaustion. For example, when the elderly person is in a hot environment, the cooperation unit increases the frequency of cooperation to prevent physical exhaustion. Furthermore, if the elderly person is in a cold environment, the cooperation unit can adjust the timing of cooperation to prevent a drop in body temperature. For example, when the elderly person is in a cold environment, the cooperation unit adjusts the timing of cooperation to prevent a decrease in body temperature. By customizing the cooperation content based on the current living conditions of the elderly person, more appropriate cooperation can be provided. Specifically, the cooperation unit receives as input vital signs obtained from wearable sensors (e.g., time-series vectors of heart rate, blood pressure, body temperature), environmental sensor data (e.g., temperature, humidity, atmospheric pressure; e.g., room temperature 28° C., humidity 70%, pressure 1010 hPa), and subjective health status reports from the elderly person (e.g., text input such as “I am not feeling well today”). The cooperation unit performs preprocessing on these multidimensional data, such as normalization, outlier removal, and feature extraction (e.g., heart rate variability indices, temperature/humidity change rates), and inputs them into a multimodal environment-adaptive cooperation decision model (e.g., a Transformer-type network integrating vital, environmental, and subjective information). Examples of input include a heart rate vector every minute (60 elements), time-series arrays of room temperature and humidity (24 hours), and health status report text sequences (e.g., “It's hot today,” “I feel chilly”). The AI model outputs cooperation intensity instruction values (e.g., 0.3=mild cooperation, 0.8=strong cooperation), cooperation frequency instruction values (e.g., 1.5 times increase), and cooperation timing adjustment values (e.g., 5 minutes earlier than usual). Examples of output include specific control parameters such as “cooperation intensity 0.3, frequency 1.5 times, timing −5 minutes.” These output values are sent to the cooperation control module and used for automatic adjustment of cooperation content according to vital signs and environmental data. For AI model training, supervised learning or transfer learning is applied using datasets of health status, environment, subjective information, and cooperation policies, and loss functions such as cross-entropy or MSE are used. Data augmentation includes adding noise to environmental data and generating paraphrases of health status reports. Unlike conventional subjective human judgment or simple threshold judgment, the cooperation unit autonomously performs pattern extraction in high-dimensional environment/health feature space and integrated analysis of multiple information using AI, greatly improving the accuracy and reproducibility of cooperation content optimization. Technical effects include improved responsiveness to health and environmental changes, prevention of excessive or insufficient cooperation, reduction of burden in care settings, and realization of environment-adaptive remote cooperation. Application fields include home care, care facilities, rehabilitation support, remote monitoring services, and personalized cooperation support.

The cooperation unit can estimate the emotion of an elderly person and determine the priority of cooperation based on the estimated emotion. For example, if the elderly person feels anxious, the cooperation unit sets the priority of cooperation high. For instance, when the elderly person feels anxious, the cooperation unit sets the priority of cooperation high. Additionally, if the elderly person is relaxed, the cooperation unit can set the priority of cooperation low. For example, when the elderly person is relaxed, the cooperation unit sets the priority of cooperation low. Furthermore, if the elderly person is in a hurry, the cooperation unit can set the priority of cooperation to the highest. For example, when the elderly person is in a hurry, the cooperation unit sets the priority of cooperation to the highest. By determining the priority of cooperation according to the emotion of the elderly person, more appropriate cooperation can be provided. Emotion estimation is realized using, for example, an emotion engine or a generative AI with emotion estimation functionality. The generative AI may be a text generation AI (e.g., LLM) or a multimodal generative AI, but is not limited to these examples. Specifically, the cooperation unit receives as input multimodal data obtained from microphones or wearable devices, such as voice data of the elderly person (e.g., 16 kHz sampling, 10-second audio waveform, content examples such as “I feel anxious today,” “I am calm”), facial image data (e.g., 640×480 pixel face images, 5 frames per second), and biometric sensor data (e.g., heart rate, skin potential, time-series vectors every minute). The cooperation unit performs preprocessing on these multimodal data, such as noise removal, normalization, and face region extraction, and extracts audio features (e.g., Mel-frequency cepstral coefficients), image features (e.g., facial expression landmark vectors), and biometric features (e.g., heart rate variability indices). The extracted features are then input to a multimodal emotion estimation model (e.g., a Transformer-type network integrating audio, image, and biometric signals), which outputs emotion labels (e.g., “anxiety,” “relaxation,” “hurry”) and emotion scores (e.g., anxiety score 0.82, relaxation score 0.15). Examples of input include a 10-second audio waveform (160,000 samples), a face image tensor of 5 frames (5×640×480×3), and a heart rate vector (10 elements). Examples of output include “Emotion label: anxiety, emotion score: 0.82” and “Emotion label: relaxation, emotion score: 0.15.” The cooperation unit sends these emotion estimation results to the cooperation priority determination module, which automatically adjusts the priority of cooperation processing with care staff, medical institutions, or family members based on rules such as “If anxiety score exceeds 0.7, set the cooperation priority to the highest,” “If relaxation score exceeds 0.8, set the priority low,” and so on. For AI model training, supervised learning or transfer learning is applied using datasets of audio, image, and biometric data labeled with emotions and cooperation priority adjustment history, and loss functions such as cross-entropy or MSE are used. Data augmentation includes adding noise to audio, rotating or changing the brightness of facial images, and time-series shifting of heart rate data. Unlike conventional subjective human observation or simple threshold judgment, the cooperation unit autonomously performs pattern extraction in high-dimensional feature space and integrated analysis of multiple modalities using AI, realizing dynamic optimization of cooperation priority according to emotional state, and greatly improving the accuracy, speed, and reproducibility of cooperation. Technical effects include optimal cooperation priority provision according to emotional state, maximization of reassurance and autonomy, rapid response in case of abnormality, reduction of burden in care settings, and realization of emotion-adaptive remote cooperation. Application fields include home care, care facilities, medical institutions, remote monitoring services, and emotion-adaptive cooperation support.

The cooperation unit can select the optimal cooperation method during cooperation by considering the geographic location information of the elderly person. For example, if the elderly person is at home, the cooperation unit focuses on cooperation around the home. For instance, when the elderly person is at home, the cooperation unit focuses on cooperation around the home. Additionally, if the elderly person is outside, the cooperation unit can focus on cooperation at the location outside. For example, when the elderly person is outside, the cooperation unit focuses on cooperation at the location outside. Furthermore, if the elderly person is in a facility, the cooperation unit can focus on cooperation within the facility. For example, when the elderly person is in a facility, the cooperation unit focuses on cooperation within the facility. By providing the optimal cooperation method based on the geographic location information of the elderly person, more appropriate cooperation can be provided. Specifically, the cooperation unit receives as input the geographic location information of the elderly person obtained from GPS modules, Wi-Fi/Bluetooth beacons, indoor location estimation sensors, etc. (e.g., latitude and longitude coordinates, labels such as home, facility, or outside, room numbers, etc.). The cooperation unit records these location data as a time series and inputs them into a geographic context estimation model (e.g., an LSTM-type network using location label sequences as input or a decision tree model using geographic features as input). Examples of input include a 24-hour location label array (e.g., “home,” “outside,” “facility”), indoor coordinate vectors (e.g., x=12.5, y=8.3), and room numbers (e.g., room 201). The AI model outputs cooperation method labels (e.g., “home area cooperation,” “outside cooperation,” “facility cooperation”) and cooperation intensity instruction values (e.g., 0.7=home cooperation, 0.9=outside cooperation). Examples of output include “Cooperation method: home area cooperation, intensity 0.7” and “Cooperation method: outside cooperation, intensity 0.9.” These output values are sent to the cooperation control module and used for automatic switching of cooperation content and frequency for vital signs and behavioral patterns. For AI model training, supervised learning or clustering is applied using datasets of location information and cooperation policy correspondence, and loss functions such as cross-entropy or MSE are used. Data augmentation includes time-series shifting of location labels and adding noise. Unlike conventional human location confirmation or simple rule-based processing, the cooperation unit autonomously performs pattern extraction in high-dimensional location feature space and integrated analysis of multiple location information using AI, greatly improving the accuracy and reproducibility of cooperation method selection. Technical effects include optimal cooperation provision according to geographic situation, prevention of fall risk and accidents, reduction of burden in care settings, and realization of location-adaptive remote cooperation. Application fields include home care, care facilities, rehabilitation support, remote monitoring services, and location-adaptive cooperation support.

The cooperation unit can analyze the social media activity of an elderly person during cooperation and provide relevant cooperation information. For example, if the elderly person shows interest in health on social media, the cooperation unit provides relevant cooperation information. For instance, when the elderly person shows interest in health on social media, the cooperation unit provides relevant cooperation information. Additionally, if the elderly person shows interest in exercise on social media, the cooperation unit can provide cooperation information related to exercise. For example, when the elderly person shows interest in exercise on social media, the cooperation unit provides cooperation information related to exercise. Furthermore, if the elderly person shows interest in diet on social media, the cooperation unit can provide cooperation information related to diet. For example, when the elderly person shows interest in diet on social media, the cooperation unit provides cooperation information related to diet. By providing relevant cooperation information based on the social media activity of the elderly person, more appropriate cooperation can be offered. Specifically, the cooperation unit acquires social media post data of the elderly person (e.g., text posts, image posts, like history, list of followed accounts, etc.) via API and inputs them into a natural language processing model (e.g., BERT or large language models) and image feature extraction models (e.g., CNN-based image classification networks). Examples of input include post texts from the past month (e.g., “I recently started walking”, “I'm paying attention to my health”), image posts (e.g., photos during exercise), like history (e.g., reactions to health-related posts), and followed accounts (e.g., celebrities, friends). The AI model outputs interest area labels (e.g., “exercise”, “health”, “diet”) and interest scores (e.g., exercise 0.85, health 0.78, diet 0.65) from these inputs. Example outputs include “Interest area: exercise, interest score 0.85”, “Interest area: health, interest score 0.78”, “Interest area: diet, interest score 0.65”. The cooperation unit sends these output values to a cooperation information recommendation module, and, for example, if the exercise interest score exceeds 0.8, cooperation information related to exercise (e.g., exercise support cooperation, exercise event notifications, etc.) is provided with emphasis; if the diet interest score exceeds 0.7, cooperation information related to diet (e.g., cooperation with nutritionists, cooperation with diet management apps, etc.) is enhanced. The content and frequency of cooperation information are automatically adjusted based on such rules. For training the AI model, supervised learning and clustering using datasets of social media activity and cooperation information recommendations are applied, and loss functions such as cross-entropy and MSE are used. Data augmentation includes paraphrase generation for post texts and rotation/brightness changes for image data. Unlike conventional manual post checking or simple keyword extraction, the cooperation unit autonomously performs pattern extraction in high-dimensional social feature space and integrated analysis of multiple post information using AI, greatly improving the accuracy, speed, and reproducibility of interest area estimation and cooperation information optimization. Technical effects include personalized cooperation information provision according to individual interests, reduction of burden in care settings, and realization of interest-adaptive cooperation from remote locations. Application fields include home care, care facilities, rehabilitation support, remote monitoring services, and personalized cooperation support.

The voice analysis unit can estimate the emotion of an elderly person and adjust the accuracy of voice analysis based on the estimated emotion. For example, if the elderly person feels anxious, the voice analysis unit increases the accuracy of voice analysis to provide a sense of security. For instance, when the elderly person feels anxious, the voice analysis unit increases the accuracy of voice analysis to provide reassurance. Additionally, if the elderly person is relaxed, the voice analysis unit can lower the accuracy to respect autonomy. For example, when the elderly person is relaxed, the voice analysis unit lowers the accuracy of voice analysis to respect autonomy. Furthermore, if the elderly person is in a hurry, the voice analysis unit can perform voice analysis quickly to support smooth analysis. For example, when the elderly person is in a hurry, the voice analysis unit performs voice analysis quickly to support smooth analysis. By adjusting the accuracy of voice analysis according to the emotion of the elderly person, more appropriate voice analysis can be provided. Emotion estimation is realized, for example, by using an emotion engine or generative AI with emotion estimation functions. Generative AI may include text generation AI (e.g., LLM) or multimodal generative AI, but is not limited to these examples.

The voice analysis unit can refer to past voice data of an elderly person during voice analysis to perform early detection of abnormalities. For example, if the elderly person has a history of abnormal breathing sounds, the voice analysis unit detects abnormal breathing sounds early. For instance, when the elderly person has a history of abnormal breathing sounds, the voice analysis unit detects abnormal breathing sounds early. Additionally, if the elderly person has a history of abnormal coughing sounds, the voice analysis unit can also detect abnormal coughing sounds early. For example, when the elderly person has a history of abnormal coughing sounds, the voice analysis unit detects abnormal coughing sounds early. Furthermore, if the elderly person has a history of abnormal speech content, the voice analysis unit can also detect abnormal speech content early. For example, when the elderly person has a history of abnormal speech content, the voice analysis unit detects abnormal speech content early. By referring to past voice data of the elderly person, early detection of abnormalities can be performed.

The voice analysis unit can customize the analysis content during voice analysis based on the current living conditions of an elderly person. For example, if the elderly person is in poor physical condition, the voice analysis unit reduces the intensity of voice analysis. For instance, when the elderly person is in poor physical condition, the voice analysis unit reduces the intensity of voice analysis. Additionally, if the elderly person is in a hot environment, the voice analysis unit can increase the frequency of voice analysis to prevent physical exhaustion. For example, when the elderly person is in a hot environment, the voice analysis unit increases the frequency of voice analysis to prevent physical exhaustion. Furthermore, if the elderly person is in a cold environment, the voice analysis unit can adjust the timing of voice analysis to prevent a drop in body temperature. For example, when the elderly person is in a cold environment, the voice analysis unit adjusts the timing of voice analysis to prevent a drop in body temperature. By customizing the analysis content based on the current living conditions of the elderly person, more appropriate voice analysis can be provided.

The voice analysis unit can estimate the emotion of an elderly person and determine the priority of voice analysis based on the estimated emotion. For example, if the elderly person feels anxious, the voice analysis unit sets the priority of voice analysis high. For instance, when the elderly person feels anxious, the voice analysis unit sets the priority of voice analysis high. Additionally, if the elderly person is relaxed, the voice analysis unit can set the priority of voice analysis low. For example, when the elderly person is relaxed, the voice analysis unit sets the priority of voice analysis low. Furthermore, if the elderly person is in a hurry, the voice analysis unit can set the priority of voice analysis to the highest. For example, when the elderly person is in a hurry, the voice analysis unit sets the priority of voice analysis to the highest. By determining the priority of voice analysis according to the emotion of the elderly person, more appropriate voice analysis can be provided. Emotion estimation is realized, for example, by using an emotion engine or generative AI with emotion estimation functions. Generative AI may include text generation AI (e.g., LLM) or multimodal generative AI, but is not limited to these examples.

The voice analysis unit can select an optimal analysis method during voice analysis by considering the geographic location information of an elderly person. For example, if the elderly person is at home, the voice analysis unit focuses on voice analysis around the home. For instance, when the elderly person is at home, the voice analysis unit focuses on voice analysis around the home. Additionally, if the elderly person is out, the voice analysis unit can focus on voice analysis at the destination. For example, when the elderly person is out, the voice analysis unit focuses on voice analysis at the destination. Furthermore, if the elderly person is in a facility, the voice analysis unit can focus on voice analysis within the facility. For example, when the elderly person is in a facility, the voice analysis unit focuses on voice analysis within the facility. By providing an optimal voice analysis method based on the geographic location information of the elderly person, more appropriate voice analysis can be provided.

The voice analysis unit can analyze the social media activity of an elderly person during voice analysis and provide relevant voice information. For example, if the elderly person shows interest in health on social media, the voice analysis unit provides relevant voice information. For instance, when the elderly person shows interest in health on social media, the voice analysis unit provides relevant voice information. Additionally, if the elderly person shows interest in exercise on social media, the voice analysis unit can provide voice information related to exercise. For example, when the elderly person shows interest in exercise on social media, the voice analysis unit provides voice information related to exercise. Furthermore, if the elderly person shows interest in diet on social media, the voice analysis unit can provide voice information related to diet. For example, when the elderly person shows interest in diet on social media, the voice analysis unit provides voice information related to diet. By providing relevant voice information based on the social media activity of the elderly person, more appropriate voice analysis can be provided.

The system according to the embodiment is not limited to the examples described above, and various modifications are possible, for example, as follows.

The operation support unit can estimate the emotion of an elderly person and adjust the intensity of operation support based on the estimated emotion. For example, if the elderly person feels anxious, the operation support unit increases the intensity of support to provide reassurance. Additionally, if the elderly person is relaxed, the operation support unit can lower the intensity of support to respect autonomy. Furthermore, if the elderly person is in a hurry, the operation support unit can provide support quickly to assist smooth operation. By adjusting the intensity of operation support according to the emotion of the elderly person, more appropriate support can be provided.

The operation support unit can analyze past exercise data of an elderly person and provide an optimal exercise plan. For example, if the elderly person has been lacking exercise in the past, the operation support unit proposes a plan to increase the amount of exercise. Additionally, if the elderly person has exercised excessively in the past, the operation support unit can propose a plan to reduce the amount of exercise. Furthermore, if the elderly person prefers a specific type of exercise, the operation support unit can provide a plan centered on that exercise. By providing an optimal exercise plan based on the past exercise data of the elderly person, more appropriate support can be provided.

The health monitoring unit can estimate the emotion of an elderly person and adjust the alert level of health monitoring based on the estimated emotion. For example, if the elderly person feels anxious, the health monitoring unit sets the alert level high and responds quickly. Additionally, if the elderly person is relaxed, the health monitoring unit can set the alert level low to avoid excessive stress. Furthermore, if the elderly person is in a hurry, the health monitoring unit can set the alert level quickly to support smooth health management. By adjusting the alert level of health monitoring according to the emotion of the elderly person, more appropriate health management can be provided.

The health monitoring unit can analyze past diet data of an elderly person and provide an optimal diet plan. For example, if the elderly person has had nutritional deficiencies in the past, the health monitoring unit proposes a nutritionally balanced diet plan. Additionally, if the elderly person has excessively consumed a specific nutrient in the past, the health monitoring unit can propose a plan to reduce that nutrient. Furthermore, if the elderly person prefers a specific food ingredient, the health monitoring unit can provide a plan centered on that ingredient. By providing an optimal diet plan based on the past diet data of the elderly person, more appropriate support can be provided.

The abnormality detection unit can estimate the emotion of an elderly person and adjust the frequency of abnormality detection based on the estimated emotion. For example, if the elderly person feels anxious, the abnormality detection unit increases the frequency of detection to provide reassurance. Additionally, if the elderly person is relaxed, the abnormality detection unit can lower the frequency of detection to respect autonomy. Furthermore, if the elderly person is in a hurry, the abnormality detection unit can perform detection quickly to support smooth response. By adjusting the frequency of abnormality detection according to the emotion of the elderly person, more appropriate abnormality detection can be provided.

The abnormality detection unit can analyze past abnormality data of an elderly person and perform early detection of abnormalities. For example, if the elderly person has a history of abnormal heart rate, the abnormality detection unit detects abnormal heart rate early. Additionally, if the elderly person has a history of abnormal blood pressure, the abnormality detection unit can also detect abnormal blood pressure early. Furthermore, if the elderly person has a history of abnormal body temperature, the abnormality detection unit can also detect abnormal body temperature early. By referring to past abnormality data of the elderly person, early detection of abnormalities can be performed.

The cooperation unit can estimate the emotion of an elderly person and adjust the frequency of cooperation based on the estimated emotion. For example, if the elderly person feels anxious, the cooperation unit increases the frequency of cooperation to provide reassurance. Additionally, if the elderly person is relaxed, the cooperation unit can lower the frequency of cooperation to respect autonomy. Furthermore, if the elderly person is in a hurry, the cooperation unit can perform cooperation quickly to support smooth response. By adjusting the frequency of cooperation according to the emotion of the elderly person, more appropriate cooperation can be provided.

The cooperation unit can analyze past cooperation data of an elderly person and provide an optimal cooperation method. For example, if the elderly person has preferred a specific cooperation method in the past, the cooperation unit prioritizes that method. Additionally, if the elderly person has had a high frequency of cooperation in the past, the cooperation unit can propose a plan to maintain that frequency. Furthermore, if the elderly person has had a low frequency of cooperation in the past, the cooperation unit can provide a plan to increase that frequency. By providing an optimal cooperation method based on the past cooperation data of the elderly person, more appropriate cooperation can be provided.

The voice analysis unit can estimate the emotion of an elderly person and adjust the frequency of voice analysis based on the estimated emotion. For example, if the elderly person feels anxious, the voice analysis unit increases the frequency of analysis to provide reassurance. Additionally, if the elderly person is relaxed, the voice analysis unit can lower the frequency of analysis to respect autonomy. Furthermore, if the elderly person is in a hurry, the voice analysis unit can perform analysis quickly to support smooth response. By adjusting the frequency of voice analysis according to the emotion of the elderly person, more appropriate voice analysis can be provided.

The voice analysis unit can analyze past voice data of an elderly person and perform early detection of abnormalities. For example, if the elderly person has a history of abnormal breathing sounds, the voice analysis unit detects abnormal breathing sounds early. Additionally, if the elderly person has a history of abnormal coughing sounds, the voice analysis unit can also detect abnormal coughing sounds early. Furthermore, if the elderly person has a history of abnormal speech content, the voice analysis unit can also detect abnormal speech content early. By referring to past voice data of the elderly person, early detection of abnormalities can be performed.

The following is a brief explanation of the processing flow of Example of the Embodiment.

Step 1: The operation support unit supports the operation of an elderly person. For example, the operation support unit provides walking assistance and supports the walking of the elderly person using a walker. Additionally, the operation support unit supports the standing-up of the elderly person using a standing-up support device. Furthermore, the operation support unit can support the operation of the elderly person using a robot.

Step 2: The conversation support unit provides conversation support based on the operation supported by the operation support unit. For example, by having a conversation while the elderly person is walking or standing up, loneliness is alleviated and a sense of security is provided. Additionally, the conversation support unit acts as a partner for daily conversation and supports the daily conversation of the elderly person.

Step 3: The health monitoring unit monitors the health condition based on the conversation supported by the conversation support unit. For example, the health monitoring unit monitors vital signs (heart rate, blood pressure, body temperature, etc.) of the elderly person in real time and learns behavioral patterns (such as wake-up time and meal time).

Step 4: The abnormality detection unit detects abnormalities based on the health condition monitored by the health monitoring unit. For example, the abnormality detection unit detects abnormalities in heart rate or blood pressure and issues an alert when the heart rate is abnormally high or low. Additionally, the abnormality detection unit detects abnormalities in behavioral patterns (such as not waking up at the usual time or not moving for a long period) and issues an alert.

Step 5: The cooperation unit cooperates with a care taxi or medical system based on the abnormality detected by the abnormality detection unit. For example, when an abnormality is detected, the cooperation unit arranges a care taxi and makes an emergency contact with a medical institution.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the above-mentioned elements, including the operation support unit, conversation support unit, health monitoring unit, abnormality detection unit, cooperation unit, and voice analysis unit, is implemented by at least one of, for example, the smart device 14 and the data processing apparatus 12. For example, the operation support unit is implemented by a control unit 46A of the smart device 14 and provides walking assistance and standing-up support for elderly people. The conversation support unit is implemented by the control unit 46A of the smart device 14 and supports daily conversation for elderly people. The health monitoring unit is implemented by a specific processing unit 290 of the data processing apparatus 12 and monitors the vital signs of elderly people in real time. The abnormality detection unit is implemented by the specific processing unit 290 of the data processing apparatus 12 and detects abnormalities in health conditions. The cooperation unit is implemented by the specific processing unit 290 of the data processing apparatus 12 and cooperates with a care taxi or medical system when an abnormality is detected. The voice analysis unit is implemented by the specific processing unit 290 of the data processing apparatus 12 and analyzes breathing sounds and coughing sounds of elderly people. The correspondence between each unit and the device or control unit is not limited to the above examples and various modifications are possible.

### Second Embodiment

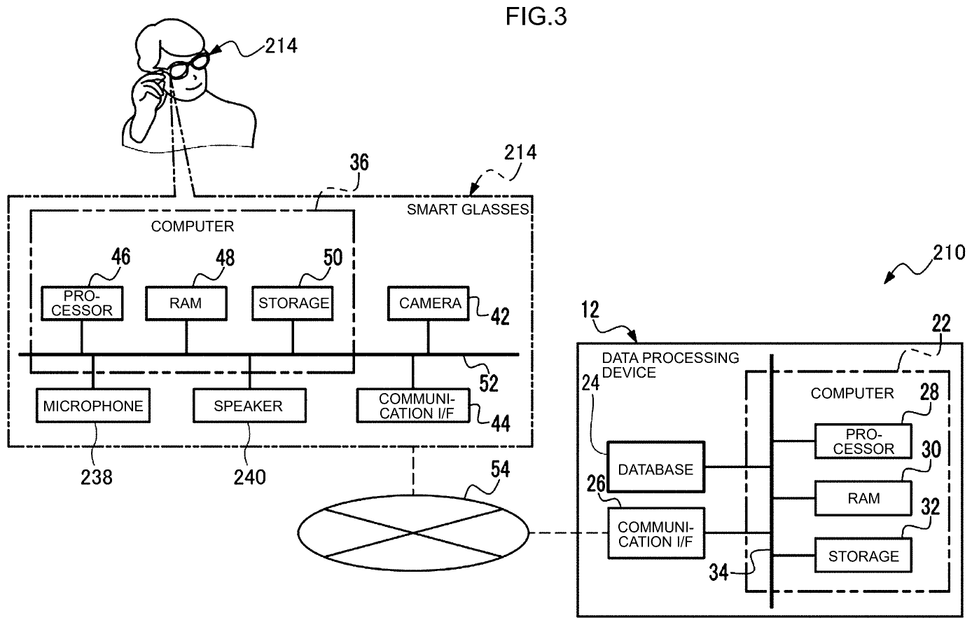

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

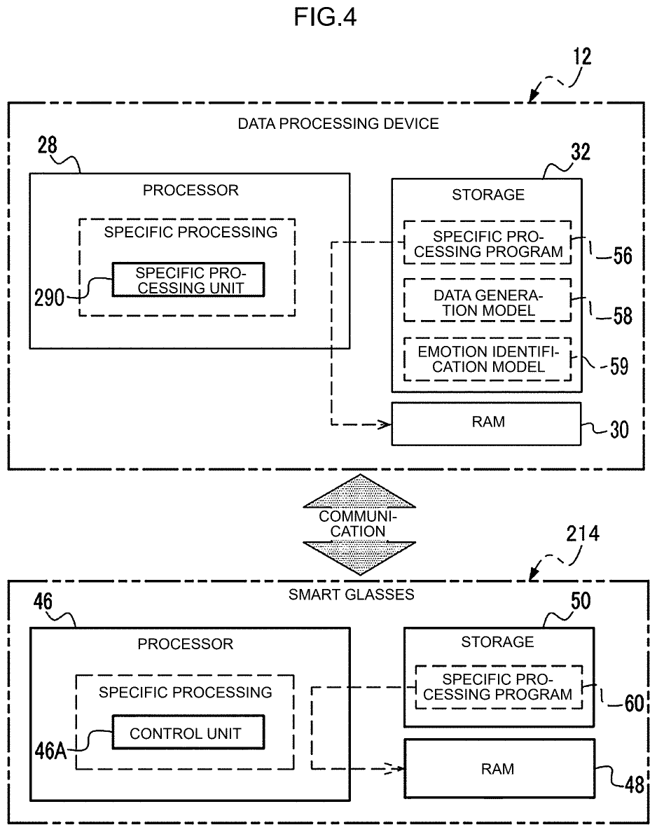

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the above-mentioned elements, including the operation support unit, conversation support unit, health monitoring unit, abnormality detection unit, cooperation unit, and voice analysis unit, is implemented by at least one of, for example, the smart glasses 214 and the data processing apparatus 12. For example, the operation support unit is implemented by a control unit 46A of the smart glasses 214 and provides walking assistance and standing-up support for elderly people. The conversation support unit is implemented by the control unit 46A of the smart glasses 214 and supports daily conversation for elderly people. The health monitoring unit is implemented by a specific processing unit 290 of the data processing apparatus 12 and monitors the vital signs of elderly people in real time. The abnormality detection unit is implemented by the specific processing unit 290 of the data processing apparatus 12 and detects abnormalities in health conditions. The cooperation unit is implemented by the specific processing unit 290 of the data processing apparatus 12 and cooperates with a care taxi or medical system when an abnormality is detected. The voice analysis unit is implemented by the specific processing unit 290 of the data processing apparatus 12 and analyzes breathing sounds and coughing sounds of elderly people. The correspondence between each unit and the device or control unit is not limited to the above examples and various modifications are possible.

### Third Embodiment

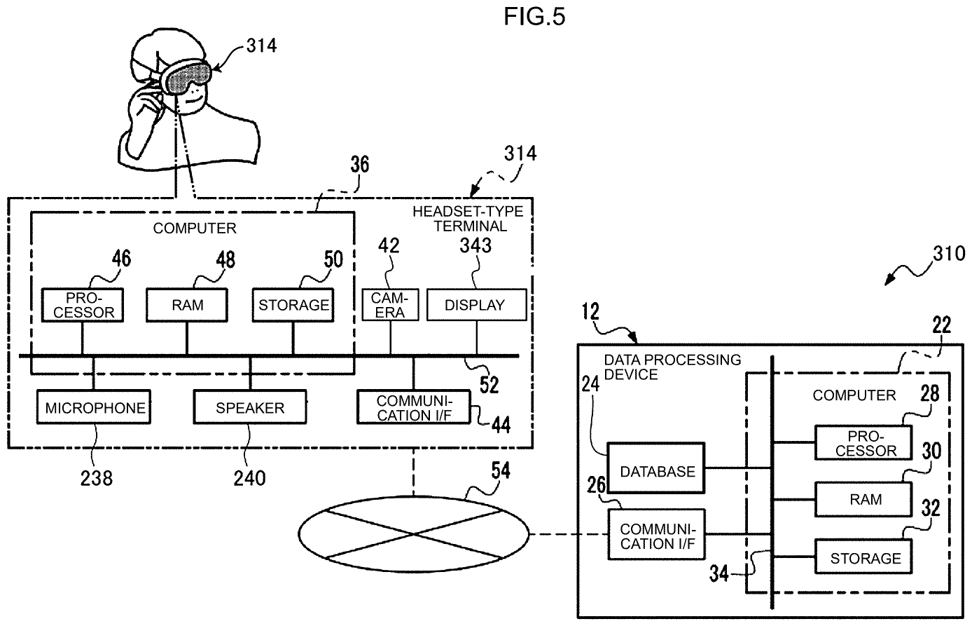

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

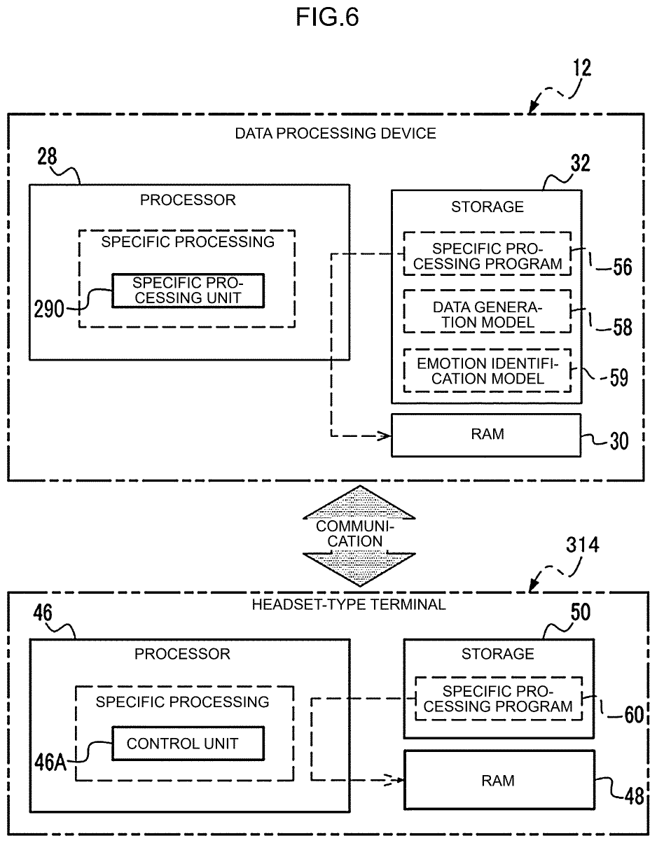

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the above-mentioned elements, including the operation support unit, conversation support unit, health monitoring unit, abnormality detection unit, cooperation unit, and voice analysis unit, is implemented by at least one of, for example, the headset-type terminal 314 and the data processing apparatus 12. For example, the operation support unit is implemented by a control unit 46A of the headset-type terminal 314 and provides walking assistance and standing-up support for elderly people. The conversation support unit is implemented by the control unit 46A of the headset-type terminal 314 and supports daily conversation for elderly people. The health monitoring unit is implemented by a specific processing unit 290 of the data processing apparatus 12 and monitors the vital signs of elderly people in real time. The abnormality detection unit is implemented by the specific processing unit 290 of the data processing apparatus 12 and detects abnormalities in health conditions. The cooperation unit is implemented by the specific processing unit 290 of the data processing apparatus 12 and cooperates with a care taxi or medical system when an abnormality is detected. The voice analysis unit is implemented by the specific processing unit 290 of the data processing apparatus 12 and analyzes breathing sounds and coughing sounds of elderly people. The correspondence between each unit and the device or control unit is not limited to the above examples and various modifications are possible.

### Fourth Embodiment

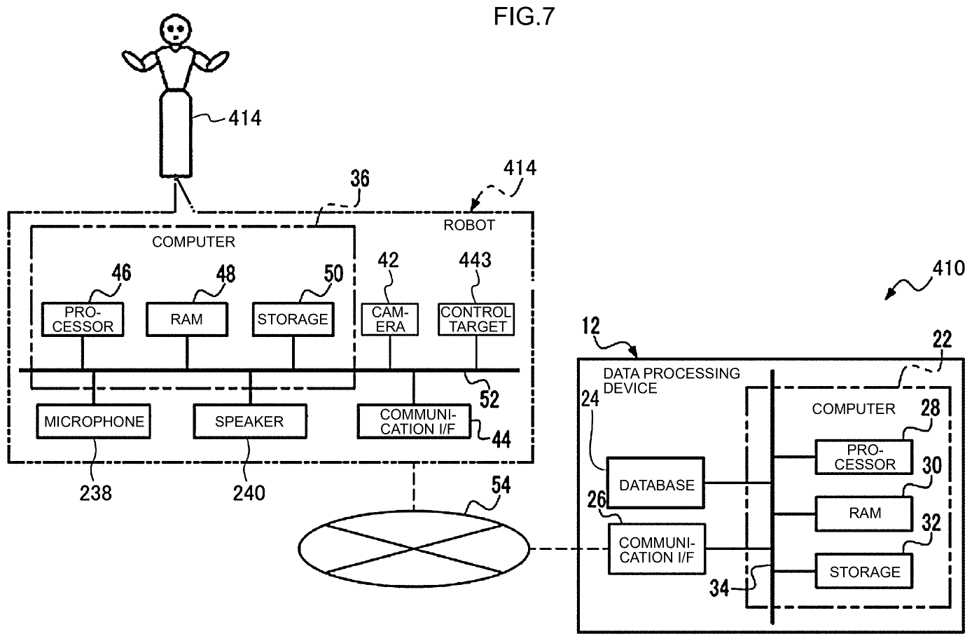

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

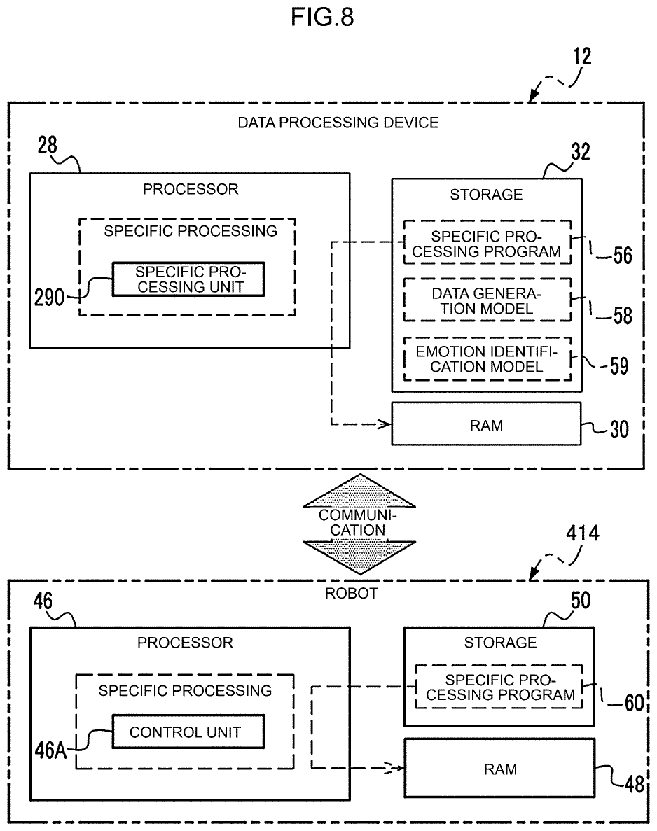

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the above-mentioned elements, including the operation support unit, conversation support unit, health monitoring unit, abnormality detection unit, cooperation unit, and voice analysis unit, is implemented by at least one of, for example, the robot 414 and the data processing apparatus 12. For example, the operation support unit is implemented by a control unit 46A of the robot 414 and provides walking assistance and standing-up support for elderly people. The conversation support unit is implemented by the control unit 46A of the robot 414 and supports daily conversation for elderly people. The health monitoring unit is implemented by a specific processing unit 290 of the data processing apparatus 12 and monitors the vital signs of elderly people in real time. The abnormality detection unit is implemented by the specific processing unit 290 of the data processing apparatus 12 and detects abnormalities in health conditions. The cooperation unit is implemented by the specific processing unit 290 of the data processing apparatus 12 and cooperates with a care taxi or medical system when an abnormality is detected. The voice analysis unit is implemented by the specific processing unit 290 of the data processing apparatus 12 and analyzes breathing sounds and coughing sounds of elderly people. The correspondence between each unit and the device or control unit is not limited to the above examples and various modifications are possible.

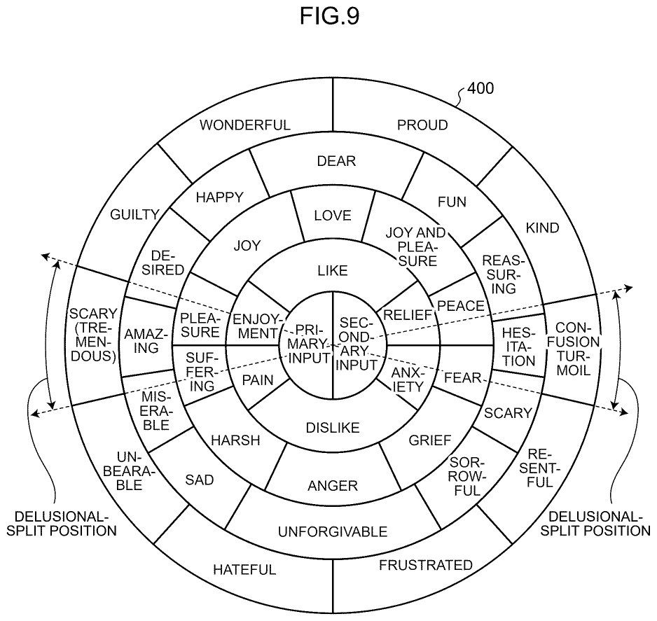

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

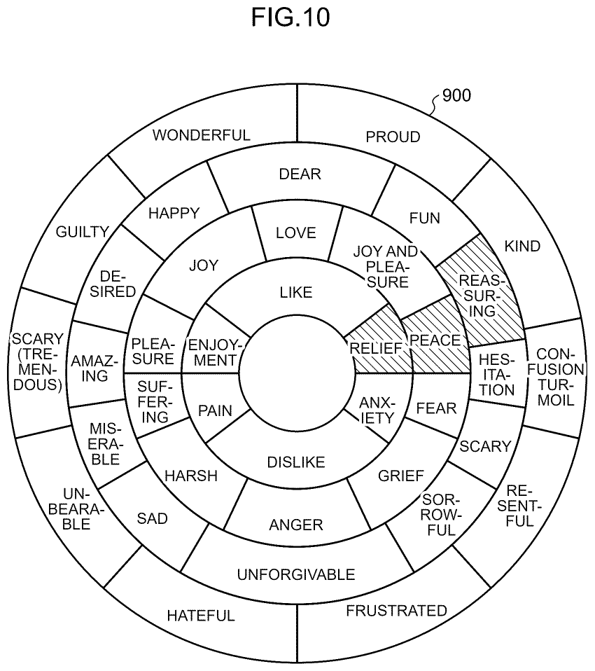

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1)A system comprising: an operation support unit; a conversation support unit configured to provide conversation support based on an operation supported by the operation support unit; a health monitoring unit configured to monitor a health condition based on a conversation supported by the conversation support unit; an abnormality detection unit configured to detect an abnormality based on the health condition monitored by the health monitoring unit; and a cooperation unit configured to cooperate with a care taxi or medical system based on the abnormality detected by the abnormality detection unit.
(Supplementary Note 2)The system according to Supplementary Note 1, wherein the operation support unit is configured to provide walking assistance.
(Supplementary Note 3)The system according to Supplementary Note 1, wherein the operation support unit is configured to provide standing-up support.
(Supplementary Note 4)The system according to Supplementary Note 1, wherein the health monitoring unit is configured to monitor vital signs in real time.
(Supplementary Note 5)The system according to Supplementary Note 1, wherein the health monitoring unit is configured to learn behavioral patterns.
(Supplementary Note 6)The system according to Supplementary Note 1, wherein the cooperation unit is configured to analyze the condition of an elderly person using voice guidance.
(Supplementary Note 7)The system according to Supplementary Note 1, wherein the cooperation unit is configured to perform necessary treatment and emergency contact.
(Supplementary Note 8)The system according to Supplementary Note 1, further comprising a voice analysis unit, wherein the voice analysis unit is configured to analyze breathing sounds and coughing sounds of an elderly person.
(Supplementary Note 9)The system according to Supplementary Note 2, wherein the voice analysis unit is configured to analyze the speech content of an elderly person.
(Supplementary Note 10)The system according to Supplementary Note 1, wherein the operation support unit is configured to estimate the emotion of an elderly person and adjust the timing of operation support based on the estimated emotion.
(Supplementary Note 11)The system according to Supplementary Note 1, wherein the operation support unit is configured to analyze the past operation history of an elderly person and select an optimal operation support method.
(Supplementary Note 12)The system according to Supplementary Note 1, wherein the operation support unit is configured to customize the support content during operation support based on the current physical condition and environment of an elderly person.
(Supplementary Note 13)The system according to Supplementary Note 1, wherein the operation support unit is configured to estimate the emotion of an elderly person and determine the priority of operation support based on the estimated emotion.
(Supplementary Note 14)The system according to Supplementary Note 1, wherein the operation support unit is configured to select an optimal support method during operation support by considering the geographic location information of an elderly person.
(Supplementary Note 15)The system according to Supplementary Note 1, wherein the operation support unit is configured to analyze the social media activity of an elderly person during operation support and provide relevant support.
(Supplementary Note 16)The system according to Supplementary Note 1, wherein the conversation support unit is configured to estimate the emotion of an elderly person and adjust the content and tone of conversation based on the estimated emotion.
(Supplementary Note 17)The system according to Supplementary Note 1, wherein the conversation support unit is configured to refer to the past conversation history of an elderly person during conversation support and provide optimal conversation content.
(Supplementary Note 18)The system according to Supplementary Note 1, wherein the conversation support unit is configured to customize the conversation content during conversation support based on the current interests and hobbies of an elderly person.
(Supplementary Note 19)The system according to Supplementary Note 1, wherein the conversation support unit is configured to estimate the emotion of an elderly person and adjust the frequency of conversation based on the estimated emotion.
(Supplementary Note 20)The system according to Supplementary Note 1, wherein the conversation support unit is configured to provide relevant topics during conversation support by considering the geographic location information of an elderly person.
(Supplementary Note 21)The system according to Supplementary Note 1, wherein the conversation support unit is configured to analyze the social media activity of an elderly person during conversation support and provide relevant topics.
(Supplementary Note 22)The system according to Supplementary Note 1, wherein the health monitoring unit is configured to estimate the emotion of an elderly person and adjust the frequency of health monitoring based on the estimated emotion.
(Supplementary Note 23)The system according to Supplementary Note 1, wherein the health monitoring unit is configured to refer to the past health data of an elderly person during health monitoring and perform early detection of abnormalities.
(Supplementary Note 24)The system according to Supplementary Note 1, wherein the health monitoring unit is configured to customize the monitoring content during health monitoring based on the current living conditions of an elderly person.
(Supplementary Note 25)The system according to Supplementary Note 1, wherein the health monitoring unit is configured to estimate the emotion of an elderly person and determine the priority of health monitoring based on the estimated emotion.
(Supplementary Note 26)The system according to Supplementary Note 1, wherein the health monitoring unit is configured to select an optimal monitoring method during health monitoring by considering the geographic location information of an elderly person.
(Supplementary Note 27)The system according to Supplementary Note 1, wherein the health monitoring unit is configured to analyze the social media activity of an elderly person during health monitoring and provide relevant health information.
(Supplementary Note 28)The system according to Supplementary Note 1, wherein the abnormality detection unit is configured to estimate the emotion of an elderly person and adjust the criteria for abnormality detection based on the estimated emotion.
(Supplementary Note 29)The system according to Supplementary Note 1, wherein the abnormality detection unit is configured to refer to the past abnormality data of an elderly person during abnormality detection and perform early detection of abnormalities.
(Supplementary Note 30)The system according to Supplementary Note 1, wherein the abnormality detection unit is configured to customize the detection content during abnormality detection based on the current living conditions of an elderly person.
(Supplementary Note 31)The system according to Supplementary Note 1, wherein the abnormality detection unit is configured to estimate the emotion of an elderly person and determine the priority of abnormality detection based on the estimated emotion.
(Supplementary Note 32)The system according to Supplementary Note 1, wherein the abnormality detection unit is configured to select an optimal detection method during abnormality detection by considering the geographic location information of an elderly person.
(Supplementary Note 33)The system according to Supplementary Note 1, wherein the abnormality detection unit is configured to analyze the social media activity of an elderly person during abnormality detection and provide relevant abnormality information.
(Supplementary Note 34)The system according to Supplementary Note 1, wherein the cooperation unit is configured to estimate the emotion of an elderly person and adjust the timing of cooperation based on the estimated emotion.
(Supplementary Note 35)The system according to Supplementary Note 1, wherein the cooperation unit is configured to refer to the past cooperation history of an elderly person during cooperation and select an optimal cooperation method.
(Supplementary Note 36)The system according to Supplementary Note 1, wherein the cooperation unit is configured to customize the cooperation content during cooperation based on the current living conditions of an elderly person.
(Supplementary Note 37)The system according to Supplementary Note 1, wherein the cooperation unit is configured to estimate the emotion of an elderly person and determine the priority of cooperation based on the estimated emotion.
(Supplementary Note 38)The system according to Supplementary Note 1, wherein the cooperation unit is configured to select an optimal cooperation method during cooperation by considering the geographic location information of an elderly person.
(Supplementary Note 39)The system according to Supplementary Note 1, wherein the cooperation unit is configured to analyze the social media activity of an elderly person during cooperation and provide relevant cooperation information.
(Supplementary Note 40)The system according to Supplementary Note 1, wherein the voice analysis unit is configured to estimate the emotion of an elderly person and adjust the accuracy of voice analysis based on the estimated emotion.
(Supplementary Note 41)The system according to Supplementary Note 1, wherein the voice analysis unit is configured to refer to the past voice data of an elderly person during voice analysis and perform early detection of abnormalities.
(Supplementary Note 42)The system according to Supplementary Note 1, wherein the voice analysis unit is configured to customize the analysis content during voice analysis based on the current living conditions of an elderly person.
(Supplementary Note 43)The system according to Supplementary Note 1, wherein the voice analysis unit is configured to estimate the emotion of an elderly person and determine the priority of voice analysis based on the estimated emotion.
(Supplementary Note 44)The system according to Supplementary Note 1, wherein the voice analysis unit is configured to select an optimal analysis method during voice analysis by considering the geographic location information of an elderly person.
(Supplementary Note 45)The system according to Supplementary Note 1, wherein the voice analysis unit is configured to analyze the social media activity of an elderly person during voice analysis and provide relevant voice information.

What is claimed is:

1. A system comprising:
circuitry configured to:
receive sensor data from a client terminal via a network;
generate, based on the sensor data, operation support data indicating a physical support operation;
transmit the operation support data to the client terminal via the network;
receive conversation data from the client terminal via the network, the conversation data being associated with the physical support operation;
generate, by inputting at least the conversation data into a data generation model, status data indicating a monitored status;
detect, based on the status data, an anomaly; and
in response to detecting the anomaly, transmit notification data to an external system via the network.

2. The system according to claim 1, wherein the physical support operation comprises a walking assistance operation.

3. The system according to claim 1, wherein the physical support operation comprises a standing-up support operation.

4. The system according to claim 1, wherein the sensor data comprises vital sign data, and wherein the circuitry is configured to monitor the vital sign data in real time.

5. The system according to claim 1, wherein the circuitry is configured to learn behavioral patterns based on the sensor data.

6. The system according to claim 1, wherein the circuitry is configured to generate voice guidance data for transmission to the client terminal in response to detecting the anomaly.

7. The system according to claim 1, wherein the external system comprises a medical system, and wherein the notification data comprises emergency contact data.

8. The system according to claim 1, wherein the circuitry is configured to receive audio data from the client terminal via the network, and analyze the audio data to detect at least one of a breathing sound abnormality or a coughing sound abnormality.

9. The system according to claim 8, wherein the circuitry is configured to analyze speech content within the audio data using a speech recognition model.

10. The system according to claim 1, wherein the circuitry is configured to estimate an emotion based on the sensor data and adjust a timing of generating the operation support data based on the estimated emotion.

11. The system according to claim 1, wherein the circuitry is configured to analyze past operation history data and select an optimal operation support method based on the past operation history data.

12. The system according to claim 1, wherein the circuitry is configured to receive environmental data from the client terminal via the network, and customize the operation support data based on the environmental data.

13. The system according to claim 1, wherein the circuitry is configured to estimate an emotion based on the conversation data and adjust a content and tone of response data based on the estimated emotion.

14. The system according to claim 1, wherein the circuitry is configured to estimate an emotion based on the status data and adjust a frequency of monitoring based on the estimated emotion.

15. The system according to claim 1, wherein the circuitry is configured to reference past status data and perform early detection of the anomaly based on the past status data.

16. The system according to claim 1, wherein the circuitry is configured to estimate an emotion and adjust criteria for detecting the anomaly based on the estimated emotion.

17. The system according to claim 1, wherein the circuitry is configured to estimate an emotion and adjust a timing of transmitting the notification data based on the estimated emotion.

18. A system comprising:
a communication interface configured to communicate with a client terminal via a network;
a processor coupled to the communication interface;
a memory storing a data generation model and an emotion identification model; and
circuitry configured to:
receive, via the communication interface, sensor data comprising acceleration data and vital sign data from the client terminal;
determine, by inputting the acceleration data into a movement recognition model, a movement state label indicating at least one of walking, standing-up, or sitting;
generate, based on the movement state label, operation support data indicating a physical support operation and a support intensity value;
transmit, via the communication interface, the operation support data to the client terminal;
receive, via the communication interface, conversation data from the client terminal;
estimate, by inputting the conversation data into the emotion identification model, an emotion label and an emotion score;
generate, by inputting the conversation data and the vital sign data into the data generation model, status data indicating a monitored status;
detect, based on the status data exceeding a threshold, an anomaly; and
in response to detecting the anomaly, transmit, via the communication interface, notification data to an external system, the notification data comprising at least an identifier, an anomaly type, and location information.

19. The system according to claim 18, wherein the circuitry is configured to receive, via the communication interface, audio data comprising breathing sounds and coughing sounds, and analyze the audio data using a convolutional neural network-based acoustic model to detect an abnormality probability and an abnormality type.

20. A method performed by circuitry of a system, the method comprising:
receiving sensor data from a client terminal via a network;
generating, based on the sensor data, operation support data indicating a physical support operation;
transmitting the operation support data to the client terminal via the network;
receiving conversation data from the client terminal via the network, the conversation data being associated with the physical support operation;
generating, by inputting at least the conversation data into a data generation model, status data indicating a monitored status;
detecting, based on the status data, an anomaly; and
in response to detecting the anomaly, transmitting notification data to an external system via the network.
